(19) United States
(12) Patent Application Publication
(10) Pub. No.: US 2026/0001951 A1
(43) Pub. Date: Jan. 1, 2026

(54) Anti-SLAMF7 Antibodies And Therapeutics

(71) Applicant: ImmunityBio, Inc., Culver City, CA (US)

(72) Inventors: Clifford Anders OLSON, Long Beach, CA (US); Marcos Sixto, Culver City, CA (US); Shiho Tanaka, Redondo Beach, CA (US)

(21) Appl. No.: 19/248,644

(22) Filed: Jun. 25, 2025

Related U.S. Application Data
(60) Provisional application No. 63/665,407, filed on Jun. 28, 2024.

(51) Int. Cl. C07K 16/28; A61K 38/20; A61K 39/00; A61K 39/395; A61P 35/00; C07K 14/54

(52) U.S. Cl. CPC C07K 16/2803; A61K 38/2086; A61K 39/39558; A61P 35/00; C07K 14/5443; A61K 2039/505; C07K 2317/31; C07K 2317/33; C07K 2317/73; C07K 2317/732; C07K 2317/92; C07K 2319/00

(57) ABSTRACT

Anti-SLAMF7 Fab fragments, antibodies, biparatopic antibodies, and chimeric antigen receptors (CARs) are provided herein. Also provided are polynucleotides encoded the anti-SLAMF7 Fab fragments, antibodies, biparatopic antibodies, and CARs. The anti-SLAMF7 Fab fragments, antibodies, biparatopic antibodies, and CARs are useful for the prevention and/or treatment of cancer, such as multiple myeloma. Also provided is a combination therapy including administration of the anti-SLAMF7 Fab fragments, antibodies, biparatopic antibodies, and CARs with an IL-16 or an IL-15 agonist.

### CROSS-REFERENCE TO RELATED PATENT APPLICATIONS

This patent application claims the benefit under 35 U.S.C. § 119(e) of U.S. Provisional Patent Application No. 63/665,407 filed on 28 Jun. 2024, the entire content of which is hereby incorporated by reference.

### FIELD

The present disclosure relates to peptides that bind to SLAMF7 and administration of anti-SLAMF7 peptides to treat cancer.

### REFERENCE TO AN ELECTRONIC SEQUENCE LISTING

This application contains references to nucleic acid sequences and/or amino acid sequences which have been submitted concurrently herewith as the sequence listing .xml file entitled “000115usps1_SequenceListing.xml”, file size 92,899 bytes, created on 30 Apr. 2024. The aforementioned sequence listing is hereby incorporated by reference in its entirety.

### BACKGROUND

This section provides background information related to the present disclosure which is not necessarily prior art.

Signaling lymphocyte activation molecule F7 (SLAMF7), also known as CD319, is a surface antigen and can be found on both normal plasma cells and malignant cells in multiple myeloma. SLAMF7 is involved in cytotoxicity, humoral immunity, autoimmunity, cell survival, and cell adhesion. SLAMF7 regulates natural killer (NK) cells' ability to recognize cancer and mediate antitumor responses. In T cells, SLAMF7 induces expression of inhibitory mechanisms related to T cell exhaustion that reduce or inhibit T cell activation. In B cells, SLAMF7 induces autocrine cytokine (e.g., IL-4) which inhibits T cell activity. SLAMF7 is highly expressed in multiple myeloma cells and is involved in its pathogenesis.

Elotuzumab, a humanized IgG1κ monoclonal antibody, and SLAMF7 CAR-T cells are examples of clinical agents for multiple myeloma. Elotuzumab targets SLAMF7 and selectively activates antibody dependent cellular cytotoxicity (ADCC) against multiple myeloma cells via NK cells, but also has been shown to improve NK cell cytotoxicity against multiple myeloma cells in a CD16-independent manner. Elotuzumab also prevents the adhesion of multiple myeloma cells to bone marrow stem cells, thus leaving the multiple myeloma cells vulnerable to cytotoxicity. Elotuzumab has shown limited clinical efficacy when used in combination with lenalidomide, pomalidomide, dexamethasone, or bortezomib. SLAMF7 CAR-T cells have been developed to address the high SLAMF7 expression of multiple myeloma cells relative to normal cells, though there are concerns about triggering fratricide among SLAMF7-expressing NK cells, B cells, and T cells. Thus, suicide genes are sometimes co-transfected into the CAR-T cell.

Zanidatamab is a bispecific, biparatopic antibody which has the antibody binding domains for both Trastuzumab and Pertuzumab which bind distinct sites on human epidermal growth factor (HER2) receptor of the EGFR family. Zanidatamab has been shown to effect antitumor activity via multiple mechanisms including tumor cell binding, HER2 internalization, cell signaling inhibition, ADCC & antibody-dependent cellular phagocytosis (ADCP), and complement dependent cytotoxicity (CDC).

Cancers grow and spread, in part, due to their ability to hide or escape from a subject's immune system. Evasion can occur through a number of mechanisms. SLAMF7, in particular, aids in a tumor's ability to evade recognition of the immune system by reducing a subject's ability to defend against cancer formation and metastasis. Despite the advances in cancer treatment, there remains a need for new and improved treatments that target cancer cells in particular.

### SUMMARY

This section provides a general summary of the disclosure and is not a comprehensive disclosure of its full scope or all of its features.

In some embodiments, the present disclosure provides antigen binding fragments (Fab fragments) or antibodies comprising a heavy variable (V_{H}) chain and a light variable (V_{L}) chain. Both the V_{H} and V_{L} chains each comprise three complementarity determining regions (CDRs) as well as non-CDR regions preceding, in between, and trailing the CDRs. The CDRs of the Fab fragments and antibodies reported herein are sufficient to bind to SLAMF7. Also reported are polynucleotide sequences that encode the anti-SLAMF7 Fab fragments or antibodies. The anti-SLAMF7 Fab fragments and antibodies, or polynucleotides that encode the anti-SLAMF7 Fab fragments or antibodies, may be formulated into a pharmaceutical composition for administration, such as intravenous administration. The pharmaceutical composition may further include one or additional agents, including, for example, a NK cell or a lysate thereof and/or a stabilized IL-15:IL-15Rα.

In other embodiments, the present disclosure provides biparatopic antibodies comprising a first arm (Arm 1) comprising a heavy variable (V_{H}) chain conjugated to a light variable (V_{L}) chain and a second arm (Arm 2) comprising an antibody heavy chain and light chains. All V_{H} and V_{L} chains each comprise three CDRs and non-CDR regions preceding, in between, and trailing the CDRs. The CDRs of the biparatopic antibodies reported herein are sufficient to bind to SLAMF7. Also reported are polynucleotide sequences that encode the anti-SLAMF7 biparatopic antibodies. The anti-SLAMF7 biparatopic antibodies, or polynucleotides that encode the anti-SLAMF7 biparatopic antibodies, may be formulated into a pharmaceutical composition for administration, such as intravenous administration. The pharmaceutical composition may further include one or additional agents, including, for example, a natural killer (NK) cell or a lysate thereof and/or a stabilized IL-15:IL-15Rα.

In other embodiments, the present disclosure provides chimeric antigen receptors (CARs) comprising a first domain, a second domain, and a third domain. The first and second domains comprise a heavy variable (V_{H}) chain and a light variable (V_{L}) chain. Both the V_{H} and V_{L} chains each comprise three CDRs and non-CDR regions preceding, in between, and trailing the CDRs. The CDRs of the CARs reported herein are sufficient to bind to SLAMF7. Also reported are polynucleotide sequences that encode the anti-SLAMF7 CAR. The anti-SLAMF7 CARs, or polynucleotides that encode the anti-SLAMF7 CAR, may be formulated into a pharmaceutical composition for administration, such as intravenous administration. The pharmaceutical composition may further include one or additional agents, including, for example, a natural killer (NK) cell or a lysate thereof and/or a stabilized IL-15:IL-15Rα.

In still another embodiment is a Fab fragment comprising a means for binding SLAMF7.

The anti-SLAMF7 Fab fragment, antibody, biparatopic antibody, CAR, or means for binding SLAMF7 may be used to treat cancer, such as multiple myeloma, in a subject in need thereof.

Various objects, features, aspects, and advantages will become more apparent from the following detailed description of preferred embodiments, along with the accompanying drawings.

### BRIEF DESCRIPTION OF THE DRAWINGS

The drawings described herein are for illustrative purposes only of selected embodiments and not all possible implementations, and are not intended to limit the scope of the present disclosure.

FIG. 1A-FIG. 1F depict the antigen dependent cellular cytotoxicity (ADCC) of anti-SLAMF7 antibodies against target HEK 293T cells that do not express SLAMF7 (FIGS. 1A, 1C, and 1E) and against target HEK 293T cells that stably express SLAMF7 (FIGS. 1B, 1D, and 1F).

FIG. 2 depicts ADCC of two anti-SLAMF7 antibodies at increasing concentrations against HEK 293T cells that stably express SLAMF7.

FIG. 3A depicts the general structure of biparatopic antibody 82-1208.

FIG. 3B depicts the general structure of biparatopic antibody 82-0812.

FIG. 4A depicts the binding of anti-SLAMF7 antibody (82-12) and biparatopic antibody (82-1208) as measured by flow cytometry.

FIG. 4B depicts the relative staining of anti-SLAMF7 antibodies (elotuzumab, 82-8, and 82-12) and anti-SLAMF7 biparatopic antibodies (82-0812 and 82-1208) relative to the mean fluorescent intensity (MFI) of antibody 82-12.

FIG. 5 depicts ADCC of biparatopic antibodies against MM1R target cells that express SLAMF7.

FIG. 6A depicts antibody dependent cellular phagocytosis (ADCP) of biparatopic antibody 82-1208-IL 15 against target MM1R cells.

FIG. 6B depicts ADCP activity of primary monocyte derived dendritic cells, M1 and M2 macrophages, and myeloid derived suppressor cells against MM1R target cells in the presence of anti-SLAMF7 positive control antibody, biparatopic antibody 82-12, and biparatopic antibody 82-1208.

FIG. 7 depicts ADCC following overnight stimulation with N-803 with primary NK cells targeting MM1R cells.

FIG. 8 depicts ADCC activity of anti-SLAMF7 bispecific IL-15 fusion.

FIGS. 9A and 9B depict CAR T cell killing assay by CAR mRNA electroporated primary T cells against HEK 293T cell stably expressing SLAMF7 or MM1R cells, respectively.

FIG. 10 depicts CAR NK cell and biparatopic antibody ADCC against MM1R target cells.

FIG. 11 depicts an in vivo experiment study design for N-803 and N-607-1208 combination therapy in a disseminated MM1R multiple myeloma model (mouse).

FIG. 12 depicts an in vivo experiment study design for N-803 and N-607-1208 combination therapy in a subcutaneous MM1R multiple myeloma model (mouse).

### DETAILED DESCRIPTION

### I. Definitions

The following definitions refer to the various terms used above and throughout the disclosure.

“Encoding”—when used to describe a polynucleotide—conveys that when transcription is initiated from the polynucleotide in a wild-type human cell, the transcript produced would be translated into a given protein. That is to say, a polynucleotide “encodes” a polypeptide when the codon triplets of wild-type human tRNA would produce the polypeptide from the polynucleotide according to the ordinary workings of transcription and translation in the wild-type human cell.

“Effective amount” or “therapeutically effective amount” refers to the amount and/or dosage, and/or dosage regime of one or more agent(s) necessary to bring about the desired result e.g., an amount sufficient to prevent an infection in a subject, an amount sufficient to reduce the occurrence of an infection in a subject, and/or an amount sufficient to treat an infection in a subject.

“IL-15 agonist” refers to a compound or molecule that binds to and activates the IL-15 receptor (“IL-15Rβγ”). The type of compound or molecule of the IL-15 agonist is not particularly limited so long as it binds to and activates the IL-15Rβγ, particularly in complex with IL-15Rα. The IL-15 agonist may be a peptide, protein, small molecule (e.g., a pharmaceutical drug), or oligonucleotide. The peptide or proteins may be a single amino acid sequence or two or more sequences bound via covalent attachments (e.g., disulfide bonds) or non-covalent attachments (e.g., hydrophilic or hydrophobic interactions, hydrogen bonds). In a particular embodiment, the IL-15 agonist is an antibody, modified antibody, chimeric antibody, or a derivative thereof. In a further embodiment, the IL-15 agonist is a superagonist complex, such as an IL-15 derivative bound to an IL-15Rα or an IL-15Rα/IgG1 Fc fusion protein, also known as nogapendekin alfa-imbakicept (NAI). NAI is also known in the literature as N-803, ALT-803, or IL-15_{N72D}:IL-15RαSu/IgG₁. U.S. Pat. No. 9,328,159, which describes NAI, is incorporated herein by reference in its entirety. Clinical trials involving N-803 are described in NCT04385849, which is incorporated herein by reference in its entirety.

Binding of the IL-15 or IL-15:IL-15Rα agonist to the IL-15Rβγ induces a signal to downstream elements to activate the IL-15 signaling pathway and activate the cell. Cells expressing IL-15Rβγ include, but are not limited to, T cells, NK cells, monocytes, macrophages, dendritic cells, keratinocytes, fibroblasts, myocytes, and nerve cells. Guo, et al. Cytokine Growth Factor Rev., 2017. Binding of IL-15 to IL-15Rα propagates a signal through IL-15Rβγ (e.g., via a conformational change) that initiates the IL-15 signaling pathway to activate an immune response, such as an antiviral response.

“Subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In certain embodiments, the subject can be human (e.g., adult male, adult female, adolescent male, adolescent female, male child, female child) under the care of a physician or other health worker. In certain embodiments the subject may not be under the care of a physician or other health worker.

An “agent that binds to SLAMF7” refers to a compound or molecule that recognizes SLAMF7 and forms a non-covalent interaction with SLAMF7 to modulate (e.g. increase or decrease) the expression and/or activity of SLAMF7. In some embodiments, the agent that binds SLAMF7 may be a Fab fragment, antibody, biparatopic antibody, or CAR.

“Treat” and “treatment” each refer to a method for reducing, inhibiting, or otherwise ameliorating an infection by administering a therapeutic to a subject in need of treatment. In some embodiments, the subject in need of treatment may include a subject having, diagnosed as having, or suspected to have, an infection. In a particular embodiment, treat or treatment include administering a therapeutic to a subject having, diagnosed as having, or suspected of having cancer, such as multiple myeloma. In some embodiments, the subject may be asymptomatic. Treatment includes administration of any of an anti-SLAMF7 Fab fragments, antibodies, biparatopic antibodies, and CARs.

### II. Antigen Binding Fragments and Antibodies

Antigen binding fragments (Fab fragments) and antibodies that bind to SLAMF7 can interfere with SLAMF7 signaling. To illustrate the anti-SLAMF7 antibodies and Fab fragments disclosed herein, exemplary Fab fragments are described having a heavy chain variable region (VII) with a structure A-V_{H} CDR1-B-V_{H} CDR2-C-V_{H} CDR3-D. These exemplary anti-SLAMF7 Fab fragments also have a light chain variable region (V_{L}) with a structure E-V_{L} CDR1-F-V_{L} CDR2-G-V_{L} CDR3-H. Amino acid sequences for the V_{H} chain and V_{L} chain of various such Fab fragments are set forth in Tables 1 and 2 below. Table 1 also shows exemplary constant regions of the V_{H} chain, including the transmembrane domain and Fc domain. Peptides comprising the V_{H} and V_{L} chains that further comprise a constant (C_{H}) region (e.g., the transmembrane domain and Fc domain) may be considered antibodies rather than Fab fragments.

TABLE 1
Exemplary anti-SLAMF7 VH sequences

| Heavy | V_{H} | | | | | | | C_{H} | |
|---|---|---|---|---|---|---|---|---|---|
| Chain | A | CDR1 | B | CDR2 | C | CDR3 | D | Transmembrane | Fc |
| 82-2 | SEQ | 1 | SEQ | 2 | SEQ | 3 | SEQ | SEQ | SEQ |
| 82-3 | ID | 4 | ID | 5 | ID | 6 | ID | ID | ID |
| 82-7 | NO: | 7 | NO:38 | 8 | NO: | 9 | NO: | NO: | NO: |
| 82-8 | 37 | 4 | | 10 | 39 | 11 | 40 | 45 | 46 |
| 82-12 | | 12 | | 5 | | 13 | | | |
| 82-28 | | 4 | | 14 | | 15 | | | |
| 82-31 | | 7 | | 16 | | 17 | | | |
| 82-36 | | 18 | | 19 | | 20 | | | |
| 82-44 | | 4 | | 21 | | 22 | | | |
| 82-25 | | 4 | | 23 | | 24 | | | |

TABLE 2
Exemplary anti-SLAMF7 VL sequences

| Light | V_{L} | | | | | | |
|---|---|---|---|---|---|---|---|
| Chain | E | CDR1 | F | CDR2 | G | CDR3 | H |
| 82-2 | SEQ | NO: | NO: | SEQ | NO: | 27 | SEQ |
| 82-3 | ID | 25 | 42 | ID | 43 | 28 | ID |
| 82-7 | NO: | | | NO: | | 29 | NO: |
| 82-8 | 41 | | | 26 | | 30 | 44 |
| 82-12 | | | | | | 31 | |
| 82-28 | | | | | | 32 | |
| 82-31 | | | | | | 33 | |
| 82-36 | | | | SEQ ID | | 34 | |
| | | | | NO: 91 | | | |
| 82-44 | | | | SEQ ID | | 35 | |
| 82-25 | | | | NO: 26 | | 36 | |

Notwithstanding the particular embodiments set out in Tables 1 and 2, the skilled person understands that the affinity of an antibody or Fab fragment is determined by the CDRs, and that a great deal of change and adjustment can be made to the non-CDR portions of the variable domains without materially altering the usefulness of these antibodies or Fab fragments for inhibiting SLAMF7. Therefore, many sequences beyond those exemplified above are equally useful for treating cancer, such as multiple myeloma. Particularly suitable sequences will retain at least 70% identity (e.g., at least 75%, at least 80%, at least 85%, at least 90%, at least 95%, at least 96%, at least 97%, at least 98%, or at least 99%) to SEQ ID NO: 37, at least 70% identity (e.g., at least 75%, at least 80%, at least 85%, at least 90%, at least 95%, at least 96%, at least 97%, at least 98%, or at least 99%) to SEQ ID NO: 38, at least 70% identity (e.g., at least 75%, at least 80%, at least 85%, at least 90%, at least 95%, at least 96%, at least 97%, at least 98%, or at least 99%) to SEQ ID NO: 39, at least 70% identity (e.g., at least 75%, at least 80%, at least 85%, at least 90%, at least 95%, at least 96%, at least 97%, at least 98%, or at least 99%) to SEQ ID NO: 40, at least 70% identity (e.g., at least 75%, at least 80%, at least 85%, at least 90%, at least 95%, at least 96%, at least 97%, at least 98%, or at least 99%) to SEQ ID NO: 41, at least 70% identity (e.g., at least 75%, at least 80%, at least 85%, at least 90%, at least 95%, at least 96%, at least 97%, at least 98%, or at least 99%) to SEQ ID NO: 42, at least 70% identity (e.g., at least 75%, at least 80%, at least 85%, at least 90%, at least 95%, at least 96%, at least 97%, at least 98%, or at least 99%) to SEQ ID NO: 43, at least 70% identity (e.g., at least 75%, at least 80%, at least 85%, at least 90%, at least 95%, at least 96%, at least 97%, at least 98%, or at least 99%) to SEQ ID NO: 44. Similarly, particular suitable sequences will retain at least 70% identity (e.g., at least 75%, at least 80%, at least 85%, at least 90%, at least 95%, at least 96%, at least 97%, at least 98%, or at least 99%) to SEQ ID NO: 45 and at least 70% identity (e.g., at least 75%, at least 80%, at least 85%, at least 90%, at least 95%, at least 96%, at least 97%, at least 98%, or at least 99%) to SEQ ID NO: 46.

In some embodiments, the V_{H} and V_{L} chains of the Fab fragments and antibodies that bind to the SLAMF7 protein may be combined as in Table 3. Each of the antibodies shown in Table 3 include a full length V_{L} chain of the general sequence E-V_{L} CDR1-F-V_{L} CDR2-G-V_{L} CDR3-H and a full length V_{H} chain of the general sequence A-V_{H} CDR1-B-V_{H} CDR2-C-V_{H} CDR3-D-transmembrane-Fc.

TABLE 3
Particular antibodies

| Antibody | V_{L} chain | V_{H} chain |
|---|---|---|
| 82-2 | SEQ ID NO: 55 | SEQ ID NO: 56 |
| 82-3 | SEQ ID NO: 57 | SEQ ID NO: 58 |
| 82-7 | SEQ ID NO: 59 | SEQ ID NO: 60 |
| 82-8 | SEQ ID NO: 61 | SEQ ID NO: 62 |
| 82-12 | SEQ ID NO: 63 | SEQ ID NO: 64 |
| 82-28 | SEQ ID NO: 69 | SEQ ID NO: 70 |
| 82-31 | SEQ ID NO: 71 | SEQ ID NO: 72 |
| 82-36 | SEQ ID NO: 73 | SEQ ID NO: 74 |
| 82-44 | SEQ ID NO: 75 | SEQ ID NO: 76 |
| 82-25 | SEQ ID NO: 67 | SEQ ID NO: 68 |

Also described herein is a Fab fragment or antibody comprising a means for binding SLAMF7 and an amino acid sequence having at least 85% sequence identity to one or more sequences selected from the group consisting of SEQ ID NOs: 37, 38, 39, 40, 41, 42, 43, and 44. The means for binding SLAMF7 relates to the combination of the three CDRs on each of the V_{H} and V_{L} chains of the antibody. Table 4 provides the combination of V_{H} and V_{L} CDRs useful for binding to SLAMF7. Sequence and functional equivalents of the combinations of Table 4 for binding SLAMF7 are also contemplated.

TABLE 4
means for binding SLAMF7

| | Structure by SEQ ID NO: | | | | | |
|---|---|---|---|---|---|---|
| Function | V_{H} CDR1 | V_{H} CDR2 | V_{H} CDR3 | V_{L} CDR1 | V_{L} CDR2 | V_{L} CDR3 |
| Binding | 1 | 2 | 3 | 25 | 26 | 27 |
| SLAMF7 | 4 | 5 | 6 | 25 | 26 | 28 |
| | 7 | 8 | 9 | 25 | 26 | 29 |
| | 4 | 10 | 11 | 25 | 26 | 30 |
| | 12 | 5 | 13 | 25 | 26 | 31 |
| | 4 | 14 | 15 | 25 | 26 | 32 |
| | 7 | 16 | 17 | 25 | 26 | 33 |
| | 18 | 19 | 20 | 25 | 91 | 34 |
| | 4 | 21 | 22 | 25 | 26 | 35 |
| | 4 | 23 | 24 | 25 | 26 | 36 |

### III. Biparatopic Antibodies

Biparatopic antibodies that bind to SLAMF7 can interfere with SLAMF7 signaling. The biparatopic antibody comprises two arms (Arm 1 and Arm 2) to bind to SLAMF7. To illustrate the biparatopic antibodies disclosed herein, exemplary biparatopic fragments comprise two arms: Arm 1 and Arm 2. Arm 1 may have a structure of V_{H}-linker-V_{L} -linker-Fc or V_{L} -linker-V_{H}-linker-Fc. In either orientation, the V_{H} and V_{L} portions may have the same structure as discussed above. For example the V_{H} portion of Arm 1 may have the structure of A-V_{H} CDR1-B-V_{H} CDR2-C-V_{H} CDR3-D and the V_{L} portion of Arm 1 may have the structure of E-V_{L} CDR1-F-V_{L} CDR2-G-V_{L} CDR3-H. Amino acid sequences for Arm 1 of exemplified biparatopic antibodies are set forth in Tables 5 and 6 below. Arm 1 or Arm 2 may further comprise an IL-15 domain or an IL-15 receptor domain (e.g., IL15RαSu).

TABLE 5
Arm 1: V_{H} - linker - V_{L} - linker - Fc

| | V_{H} (SEQ ID NOs) | | | | | | | | V_{L} (SEQ ID NOs) | | | | | | | | |
|---|---|---|---|---|---|---|---|---|---|---|---|---|---|---|---|---|---|
| Arm | | CDR | | CDR | | CDR | | | | CDR | | CDR | | CDR | | | |
| 1 | | 1 | | 2 | | 3 | | | | 1 | | 2 | | 3 | | | Fc |
| 82- | SEQ | 1 | SEQ | 5 | SEQ | 13 | SEQ | Linker | SEQ | SEQ | SEQ | SEQ | SEQ | 31 | SEQ | Linker | SEQ |
| 1208 | ID | 4 | ID | 10 | ID | 11 | ID | | ID | ID | ID | ID | ID | 30 | ID | | ID |
| 82- | NO: | 12 | NO: | 5 | NO: | 13 | NO: | | NO: | NO: | NO: | NO: | NO: | 31 | NO: | | NO: |
| 0812 | 37 | | 38 | | 39 | | 40 | | 41 | 25 | 42 | 26 | 43 | | 44 | | 46 |
| 82- | | | | | | | | | | | | | | | | | |
| 0812* | | | | | | | | | | | | | | | | | |

*includes IL 15RαSu domain (SEQ ID NO: 49)

TABLE 6
Arm 1: V_{L} - linker - V_{H} - linker - Fc

| | V_{H} (SEQ ID NOs) | | | | | | | | V_{L} (SEQ ID NOs) | | | | | | | | |
|---|---|---|---|---|---|---|---|---|---|---|---|---|---|---|---|---|---|
| | | CDR | | CDR | | CDR | | | | CDR | | CDR | | CDR | | | |
| Arm 1 | | 1 | | 2 | | 3 | | Linker | | 1 | | 2 | | 3 | | Linker | Fc |
| 82-LH | 41 | 25 | 42 | 26 | 43 | 31 | 44 | | 37 | 12 | 38 | 5 | 39 | 13 | 40 | | 46 |
| 1208 | | | | | | | | | | | | | | | | | |

The linker in Arm 1 is not particularly limited. In some embodiments, the linker may be a poly-glycine linker comprising four or more glycines. One or more of the glycines in the linker may be replaced with another amino acids. Particular linkers include, independently, GGGGSGGGGSGGGGSGGGG (SEQ ID NO: 51), GGGGSKGGGGSKGGG (SEQ ID NO: 52), GGGGSGGGGSGGGGS (SEQ ID NO: 53), or GGGGSGGGGSGGGGSGGGGSGGGGS GGGGS (SEQ ID NO: 54).

Arm 2 may have both a V_{L} chain and V_{H} chain wherein the V_{L} chain has the structure of E-V_{L} CDR1-F-V_{L} CDR2-G-V_{L} CDR3-H and the V_{H} chain has the structure of A-V_{H} CDR1-B-V_{H} CDR2-C-V_{H} CDR3-D covalently linked to a transmembrane domain-Fc domain. The V_{L} and V_{H} chains are linked to each other via disulfide bonds between the variable regions of each chain. Amino acid sequences for the V_{H} chain and V_{L} chain of Arm 2 are set forth in Table 7.

TABLE 7
Arm 2: LC + HC (- linker - IL-15)

| | Light Chain (LC) | | | | | | | Heavy Chain (HC) | | | | | | | | |
|---|---|---|---|---|---|---|---|---|---|---|---|---|---|---|---|---|
| | | CDR | | CDR | | CDR | | | CDR | | CDR | | CDR | | | |
| Arm 2 | | 1 | | 2 | | 3 | | | 1 | | 2 | | 3 | | TM | Fc |
| 82-1208 | SEQ | SEQ | SEQ | SEQ | SEQ | 30 | SEQ | SEQ | 4 | SEQ | 10 | SEQ | 11 | SEQ | SEQ | SEQ |
| 82-LH | ID | ID | ID | ID | ID | 30 | ID | ID | 4 | ID | 10 | ID | 11 | ID | ID | ID |
| 1208 | NO: | NO: | NO: | NO: | NO: | 31 | NO: | NO: | 12 | NO: | 5 | NO: | 13 | NO: | NO: | NO: |
| 82-0812 | 41 | 25 | 42 | 26 | 43 | 30 | 44 | 37 | 4 | 38 | 10 | 39 | 11 | 40 | 45 | 46 |
| 82- | | | | | | | | | | | | | | | | |
| 0812\hat{ } | | | | | | | | | | | | | | | | |

\hat{ }includes wt IL-15 domain (SEQ ID NO: 47) or N72D IL-15 domain (SEQ ID NO: 48)

Notwithstanding the particular embodiments set out in Tables 5-7, the skilled person understands that the affinity of a biparatopic antibody is determined by CDRs, and that a great deal of change and adjustment can be made to the non-CDR portions of the variable domains without materially altering the usefulness of the biparatopic antibodies for inhibiting SLAMF7. Therefore, many sequences beyond those exemplified above are equally useful for treating cancer, such as multiple myeloma. Particularly suitable sequences will retain at least 70% identity (e.g., at least 75%, at least 80%, at least 85%, at least 90%, at least 95%, at least 96%, at least 97%, at least 98%, or at least 99%) to SEQ ID NO: 37, at least 70% identity (e.g., at least 75%, at least 80%, at least 85%, at least 90%, at least 95%, at least 96%, at least 97%, at least 98%, or at least 99%) to SEQ ID NO: 38, at least 70% identity (e.g., at least 75%, at least 80%, at least 85%, at least 90%, at least 95%, at least 96%, at least 97%, at least 98%, or at least 99%) to SEQ ID NO: 39, at least 70% identity (e.g., at least 75%, at least 80%, at least 85%, at least 90%, at least 95%, at least 96%, at least 97%, at least 98%, or at least 99%) to SEQ ID NO: 40, at least 70% identity (e.g., at least 75%, at least 80%, at least 85%, at least 90%, at least 95%, at least 96%, at least 97%, at least 98%, or at least 99%) to SEQ ID NO: 41, at least 70% identity (e.g., at least 75%, at least 80%, at least 85%, at least 90%, at least 95%, at least 96%, at least 97%, at least 98%, or at least 99%) to SEQ ID NO: 42, at least 70% identity (e.g., at least 75%, at least 80%, at least 85%, at least 90%, at least 95%, at least 96%, at least 97%, at least 98%, or at least 99%) to SEQ ID NO: 43, at least 70% identity (e.g., at least 75%, at least 80%, at least 85%, at least 90%, at least 95%, at least 96%, at least 97%, at least 98%, or at least 99%) to SEQ ID NO: 44. Similarly, particular suitable sequences will retain at least 70% identity (e.g., at least 75%, at least 80%, at least 85%, at least 90%, at least 95%, at least 96%, at least 97%, at least 98%, or at least 99%) to SEQ ID NO: 45 and at least 70% identity (e.g., at least 75%, at least 80%, at least 85%, at least 90%, at least 95%, at least 96%, at least 97%, at least 98%, or at least 99%) to SEQ ID NO: 46.

Alternatively and additionally, the biparatopic antibody may further comprise an IL-15 domain or an IL-15 receptor domain. The IL-15 may be wildtype IL-15 (e.g., SEQ ID NO: 47) or an N72D mutant IL-15 (e.g., SEQ ID NO: 48). The IL-15 domain may retain at least 70% sequence identity (e.g., at least 75%, at least 80%, at least 85%, at least 90%, at least 95%, at least 96%, at least 97%, at least 98%, or at least 99%) to SEQ ID NO: 47 or SEQ ID NO: 48. The IL-15 domain may be conjugated to Arm 1 or Arm 2, for example at the C-terminal end of Arm 1 or Arm 2 via a linker. The biparatopic antibody may further comprise an IL-15 receptor, for example an IL15RαSu. The IL15RαSu may have the amino acid sequence of SEQ ID NOs: 49. The IL-15 receptor domain may retain at least 70% sequence identity (e.g., at least 75%, at least 80%, at least 85%, at least 90%, at least 95%, at least 96%, at least 97%, at least 98%, or at least 99%) to SEQ ID NO: 49. The IL-15 receptor domain may be conjugated to Arm 1 or Arm 2, for example at the C-terminal end of Arm 1 or Arm 2 via a linker.

In some embodiments, the V_{H} and V_{L} chains of the biparatopic antibodies that bind to the SLAMF7 protein may be combined as in Table 8. Each of the biparatopic antibodies shown in Table 8 include Arm 1 and Arm 2, which includes both a V_{L} and V_{H} chain, as shown above. Biparatopic antibody 82-0812-IL-15 comprises IL15RαSu (SEQ ID NO: 49) conjugated via a linker to the C-terminal end of Arm 1 and an N72D IL-15 conjugated via a linker to the C-terminal end of the Arm 2 heavy chain.

TABLE 8
Particular biparatopic antibodies

| | Arm 1 | Arm 2 LC | Arm 2 HC |
|---|---|---|---|
| Biparatopic Antibody | SEQ ID NO | SEQ ID NO | SEQ ID NO |
| 82-1208 | 77 | 78 | 79 |
| 82-0812 | 83 | 84 | 85 |
| 82-0812-IL-15 | 86 | 87 | 88 |
| 82-LH1208 | 80 | 81 | 82 |

Alternatively, the biparatopic antibody may comprise a means for binding SLAMF7 and an amino acid sequence having at least 85% sequence identity to one or more sequences selected from the group consisting of SEQ ID NOs: 37, 38, 39, 40, 41, 42, 43, and 44. The means for binding SLAMF7 relates to the combination of the three CDRs on each of the V_{H} and V_{L} chains of Arm 1 and Arm 2 of the biparatopic antibody. Tables 9 and 10 provide the combination of V_{H} and V_{L} CDRs on Arm 1 and Arm 2, respectively, useful for binding to SLAMF7. Sequence and functional equivalents of the combinations of Tables 9 and 10 for binding SLAMF7 are also contemplated.

TABLE 9
means for binding SLAMF7 (Arm 1)

| | Structure by SEQ ID NO: | | | | | |
|---|---|---|---|---|---|---|
| Function | V_{H} CDR1 | V_{H} CDR2 | V_{H} CDR3 | V_{L} CDR1 | V_{L} CDR2 | V_{L} CDR3 |
| Binding | 1 | 2 | 3 | 25 | 26 | 27 |
| SLAMF7 | 4 | 5 | 6 | 25 | 26 | 28 |
| | 7 | 8 | 9 | 25 | 26 | 29 |
| | 4 | 10 | 11 | 25 | 26 | 30 |
| | 12 | 5 | 13 | 25 | 26 | 31 |
| | 4 | 14 | 15 | 25 | 26 | 32 |
| | 7 | 16 | 17 | 25 | 26 | 33 |
| | 18 | 19 | 20 | 25 | 91 | 34 |
| | 4 | 21 | 22 | 25 | 26 | 35 |
| | 4 | 23 | 24 | 25 | 26 | 36 |

TABLE 10
means for binding SLAMF7 (Arm 2)

| | Structure by SEQ ID NO: | | | | | |
|---|---|---|---|---|---|---|
| Function | V_{H} CDR1 | V_{H} CDR2 | V_{H} CDR3 | V_{L} CDR1 | V_{L} CDR2 | V_{L} CDR3 |
| Binding | 1 | 2 | 3 | 25 | 26 | 27 |
| SLAMF7 | 4 | 5 | 6 | 25 | 26 | 28 |
| | 7 | 8 | 9 | 25 | 26 | 29 |
| | 4 | 10 | 11 | 25 | 26 | 30 |
| | 12 | 5 | 13 | 25 | 26 | 31 |
| | 4 | 14 | 15 | 25 | 26 | 32 |
| | 7 | 16 | 17 | 25 | 26 | 33 |
| | 18 | 19 | 20 | 25 | 91 | 34 |
| | 4 | 21 | 22 | 25 | 26 | 35 |
| | 4 | 23 | 24 | 25 | 26 | 36 |

### IV. Chimeric Antigen Receptors (CAR)

CARs that bind to SLAMF7 can interfere with SLAMF7 signaling. To illustrate the anti-SLAMF7 CARs disclosed herein, exemplary CARs are described having three domains. The first domain may comprise a V_{H} chain or a VI, chain and the second domain may comprise a V_{H} chain and a V_{L} chain, if the first domain comprises a V_{H} chain, the second domain comprises a V_{L} chain, or vice versa. The first domain and second domain are conjugated via a linker. The third domain may be directly conjugated to the second domain.

The V_{H} of the of the CAR comprises three CDRs and has the general structure A-V_{H} CDR1-B-V_{H} CDR2-C-V_{H} CDR3-D. The V_{L} chain of the CAR comprises three CDRs and has the general structure E-V_{L}CDR1-F-V_{L} CDR2-G-V_{L} CDR3-H. Amino acid sequences for the V_{H} chain and V_{L} chain of the CAR are set forth in Tables 1 and 2 above.

Notwithstanding the particular embodiments set out in Tables 1 and 2, the skilled person understands that the affinity of a CAR is determined by the complementarity determining regions (CDRs), and that a great deal of change and adjustment can be made to the non-CDR portions of the CAR without materially altering the usefulness of these CAR for inhibiting SLAMF7. Therefore, many sequences beyond those exemplified above are equally useful for treating cancer, such as multiple myeloma. Particularly suitable sequences will retain at least 70% identity (e.g., at least 75%, at least 80%, at least 85%, at least 90%, at least 95%, at least 96%, at least 97%, at least 98%, or at least 99%) to SEQ ID NO: 37, at least 70% identity (e.g., at least 75%, at least 80%, at least 85%, at least 90%, at least 95%, at least 96%, at least 97%, at least 98%, or at least 99%) to SEQ ID NO: 38, at least 70% identity (e.g., at least 75%, at least 80%, at least 85%, at least 90%, at least 95%, at least 96%, at least 97%, at least 98%, or at least 99%) to SEQ ID NO: 39, at least 70% identity (e.g., at least 75%, at least 80%, at least 85%, at least 90%, at least 95%, at least 96%, at least 97%, at least 98%, or at least 99%) to SEQ ID NO: 40, at least 70% identity (e.g., at least 75%, at least 80%, at least 85%, at least 90%, at least 95%, at least 96%, at least 97%, at least 98%, or at least 99%) to SEQ ID NO: 41, at least 70% identity (e.g., at least 75%, at least 80%, at least 85%, at least 90%, at least 95%, at least 96%, at least 97%, at least 98%, or at least 99%) to SEQ ID NO: 42, at least 70% identity (e.g., at least 75%, at least 80%, at least 85%, at least 90%, at least 95%, at least 96%, at least 97%, at least 98%, or at least 99%) to SEQ ID NO: 43, at least 70% identity (e.g., at least 75%, at least 80%, at least 85%, at least 90%, at least 95%, at least 96%, at least 97%, at least 98%, or at least 99%) to SEQ ID NO: 44. Similarly, particular suitable sequences will retain at least 70% identity (e.g., at least 75%, at least 80%, at least 85%, at least 90%, at least 95%, at least 96%, at least 97%, at least 98%, or at least 99%) to SEQ ID NO: 50 in the third domain.

In some embodiments, the V_{H} and V_{L} chains of the CAR that bind to the SLAMF7 protein may be combined as in Table 11 Each of the CARs shown in Table 11 include a full length V_{H} chain of the general sequence A-V_{H} CDR1-B-V_{H} CDR2-C-V_{H} CDR3-D and a full length V_{L} chain of the general sequence E-V_{L} CDR1-F-V_{L} CDR2-G-V_{L} CDR3-H and in the first domain or the second domain, respectively. Each of the CARs of Table 11 comprise a third domain having the amino acid sequence according to SEQ ID NO: 50. Preferred CARs have the amino acid sequence according to SEQ ID NO: 89 or SEQ ID NO: 90.

TABLE 11
CAR

| | First Domain | | | Second Domain | | |
|---|---|---|---|---|---|---|
| | (SEQ ID NO) | | | (SEQ ID NO) | | |
| | CDR1 | CDR2 | CDR3 | CDR1 | CDR2 | CDR3 |
| 82-2 CAR | 1 | 2 | 3 | 25 | 26 | 27 |
| 82-3 CAR | 4 | 5 | 6 | 25 | 26 | 28 |
| 82-7 CAR | 7 | 8 | 9 | 25 | 26 | 29 |
| 82-8 CAR | 4 | 10 | 11 | 25 | 26 | 30 |
| 82-12-CAR | 12 | 5 | 13 | 25 | 26 | 31 |
| 82-28 CAR | 4 | 14 | 15 | 25 | 26 | 32 |
| 82-31 CAR | 7 | 16 | 17 | 25 | 26 | 33 |
| 82-36 CAR | 18 | 19 | 20 | 25 | 91 | 34 |
| 82-44 CAR | 4 | 21 | 22 | 25 | 26 | 35 |
| 82-25 CAR | 4 | 23 | 24 | 25 | 26 | 36 |

Alternatively, the CAR may comprise a means for binding SLAMF7 and an amino acid sequence having at least 85% sequence identity to one or more sequences selected from the group consisting of SEQ ID NOs: 37, 38, 39, 40, 41, 42, 43, and 44. The means for binding SLAMF7 relates to the combination of the three CDRs on each of the V_{H} and v_{L} chains of the first and second domains of the CAR. Table 4, above, provides the combination of V_{H} and V_{L} CDRs within the first and second domains, respectively, useful for binding to SLAMF7. Sequence and functional equivalents of the combinations of Table 4 for binding SLAMF7 are also contemplated.

### V. Polynucleotides and Vectors

Contemporary molecular biologists know how to make nucleotides that express the proteins described herein, and how to express such nucleotides in cells to obtain the relevant proteins. Further embodiments provided herein include polynucleotides that encode a polypeptide comprising anti-SLAMF7 Fab fragments, antibodies, biparatopic antibodies, and CARs.

In certain embodiments, the polynucleotides described above can be expressed in a supporter cell line. Mammalian cell lines, such as Chinese hamster ovary (CHO) cells or 293T cells, are particularly suitable for these purposes. The proteins described above are generally soluble and will therefore be excreted from a producing cell unless they are modified for intracellular retention. Proteins produced in this manner can be purified from the culture medium. Where desired, the proteins may be tagged with, for example, a poly-histidine tag or other such commercially common tags to facilitate purification. Proteins produced and purified in this manner can then be administered to a subject in need thereof as described below.

### VI. Pharmaceutical Compositions

The proteins, polynucleotides, and vectors described above can be used to treat, reduce the occurrence of, and/or prevent cancer, particularly multiple myeloma, via formulation in a pharmaceutical composition. Thus, in some embodiments the pharmaceutical composition may be for use in preventing, reducing the occurrence of, and/or treating cancer, such as multiple myeloma. The pharmaceutical composition comprises the anti-SLAMF7 Fab fragment, antibody, biparatopic antibody, or CAR described herein and further may comprise one or more additional active agents or compositions and/or pharmaceutically acceptable excipients.

In an embodiment, the one or more additional active composition may be an NK cell or a lysate thereof. An NK cell may be a cell line such as an NK-92 line or a genetically engineered derivative thereof, or a primary NK cell such as a memory-like cytokine enhanced NK (m-ceNK) cell. An NK cell lysate refers to a lysate generated by lysing at least one NK cell. An NK cell lysate may be prepared from at least one NK cell, where the lysate lacks NK cell membranes. Such an NK cell lysate is prepared from NK cells that have been lysed, i.e., NK cells in which the cell membranes have been disrupted, exposing NK cell contents to the rest of the composition. An NK cell may be lysed by known methods, such as by electrical, physical, chemical, and enzymatic techniques. Example disruption methods include high pressure or liquid homogenization, ultrasonication, sonication, freeze-thaw, and manual grinding, or detergent/solution-based cell lysis.

Exemplary NK cell lysates are described in U.S. Application No. 63/635,805, which is herein incorporated by reference. The NK cell lysate may comprise a salt solution, cytotoxic protein, and one or more cytokines. The NK cell lysate may be derived from NK-92/aNK cells, haNK cells, primary NK cells, KHYG-1 cells, or a variant thereof. The cytokine may be one or more of include IL-1α, IL-1β, IL-2, IL-4, IL-5, IL-6, IL-7, IL-10, IL-11, IL-12p40, IL-12p70, IL-13, IL-15, IL-16, IL-17, INF-γ, GM-CSF, G-CSF, TNF-α, TNF-β, VEGF, IL-8, Eotaxin-3, MCP-1, MCP-4, MDC, MIP-1α, MIP-1β, and TARC. In a particular embodiment, the cytokine is IL-16 which may be purified from an NK cell lysate.

Additionally or alternatively, the one or more additional active agent may be an IL-15 agonist. In a particular embodiment, the IL-15 agonist is an antibody, modified antibody, chimeric antibody, or a derivative thereof. In a further embodiment, the IL-15 agonist is a superagonist complex, such as an IL-15 derivative bound to an IL-15Rα/IgG1 Fc fusion protein, also known as nogapendekin alfa-imbakicept (NAI). NAI is also known in the literature as N-803, ALT-803, or IL-15_{N72D}:IL-15RαSu/IgG₁. U.S. Pat. No. 9,328,159, which describes NAI, is incorporated herein by reference in its entirety. IL-15 superagonists typically comprise IL-15:IL-15RαSu complexes. Clinical trials involving N-803 are described in NCT04385849, which is incorporated herein by reference in its entirety. IL-15 agonists such as N-803 may be included in the administration of a SLAMF7 Ab to address Ab treatment associated lymphopenia.

In an embodiment, a pharmaceutical composition may comprise at least one checkpoint inhibitor Ab selected from the group consisting of a PD-L1Ab, a PD-1 Ab, a TIM-3 inhibitor, and a CTLA-4 Ab. The pharmaceutical composition may comprise an HDAC inhibitor. The pharmaceutical composition may comprise a proteosome inhibitor.

In some embodiments, it may be beneficial to include one or more excipient in a composition. One of skill in the art would appreciate that the choice of any one excipient may influence the choice of any other excipient. For example, the choice of a particular excipient may preclude the use of one or more additional excipients because the combination of excipients would produce undesirable effects. One of skill in the art would be able to determine empirically which excipients, if any, to include in the formulations or compositions disclosed herein. Excipients may include, but are not limited to, co-solvents, solubilizing agents, buffers, pH adjusting agents, bulking agents, surfactants, encapsulating agents, tonicity-adjusting agents, stabilizing agents, protectants, and viscosity modifiers. In some embodiments, it may be beneficial to include a pharmaceutically acceptable carrier.

The pharmaceutical composition comprising the anti-SLAMF7 Fab fragment, antibody biparatopic antibody, or CARs may be formulated for its intended route of administration. For example, the composition may be formulated for systemic, peripheral, proximal to the location of a target site (e.g., near a tumor), and/or intratumorally. Suitable routes of administration will be apparent to those of skill in the art, depending on the type of condition to be prevented or treated, the therapeutically active agent used, and/or the target cell population or tissue. Various acceptable methods of administration include, but are not limited to, intravenous administration, intraperitoneal administration, intramuscular administration, intranodal administration, intracoronary administration, intraarterial administration (e.g., into a carotid artery), subcutaneous administration, retroorbital administration, transdermal delivery, intratracheal administration, subcutaneous administration, intraarticular administration, intraventricular administration, inhalation (e.g., aerosol), intracranial, intraspinal, intraocular, aural, intranasal, oral, pulmonary administration, impregnation of a catheter, and direct injection into a tissue. In one aspect, routes of administration include: intravenous, intraperitoneal, subcutaneous, intradermal, intranodal, intramuscular, transdermal, inhaled, intranasal, oral, intraocular, intraarticular, intracranial, and intraspinal. Parenteral delivery can include intradermal, intramuscular, intraperitoneal, intrapleural, intrapulmonary, intravenous, subcutaneous, atrial catheter and venal catheter routes. Aural delivery can include ear drops, intranasal delivery can include nose drops or intranasal injection, and intraocular delivery can include eye drops. Aerosol (inhalation) delivery can also be performed using methods standard in the art. Other routes of administration that modulate mucosal immunity are useful in the treatment of viral infections. Such routes include bronchial, intradermal, intramuscular, intranasal, other inhalatory, rectal, subcutaneous, topical, transdermal, vaginal and urethral routes. In a particular embodiment the pharmaceutical composition is administered intravenously.

### VII. Methods of Treatment

The proteins, polynucleotides, and vectors described above can be used to treat and/or prevent cancer, particularly multiple myeloma. To treat and/or prevent this disease, the proteins, polynucleotides, and/or vectors described above may be administered to a subject in need thereof in a therapeutically effective amount. The subject may be symptomatic or asymptomatic.

Where an anti-SLAMF7 Fab fragment, antibody, biparatopic antibody, or CAR (or a pharmaceutical composition comprising the same) is to be administered, any suitable route of administration may be used, including but not limited to intravenous injection, intramuscular injection, subcutaneous injection, and inhalation (e.g. aerosol inhalation). Therapeutically effective amounts of these proteins include but are not limited to about 1 μg of protein per kg of subject body weight, about 5 μg/kg, about 10 μg/kg, about 50 μg/kg, about 100 μg/kg, about 500 μg/kg, about 1 mg/kg, about 5 mg/kg, about 10 mg/kg, about 50 mg/kg, about 100 mg/kg, about 500 mg/kg, and about 1 mg/kg or more. Alternatively, the anti-SLAMF7 Fab fragment, antibody, biparatopic antibody, or CAR can be administered in an amount independent of the subject's mass or weight. For example, a therapeutically effective amount of the anti-SLAMF7 Fab fragment, antibody, biparatopic antibody, or CAR may be about 10 mg, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,050 mg, about 1,100 mg, about 1,200 mg, about 1,250 mg, about 1,300 mg, about 1,350 mg, about 1,400 mg, about 1,450 mg, and about 1,500 mg or more.

Where a polynucleotide is to be transfected, any suitable amount can be administered, including (but not limited to) 10 ng, 50 ng, 100 ng, 500 ng, 1 μg, 5 μg, 10 μg, 50 μg, 100 μg, 500 μg, 1 mg, 5 mg, 10 mg, 50 mg, 100 mg, and 500 mg or more. To transfect subject cells with polynucleotides as described herein, it will be useful to extract cells from the subject, transfect them according to known techniques, and then transfuse the transfected cells back into the subject. Particularly suitable cells circulate throughout the body, such as circulating lymphocytes.

Where a polynucleotide is to be transduced, the viral vector can be administered directly to the subject, or cells can be extracted for transduction and re-transfusion. The viral vector can be administered to the subject by any suitable route of administration, including but not limited to intravenous injection, intramuscular injection, subcutaneous injection, and inhalation (e.g. aerosol inhalation). In a particular embodiment, the viral vector is administered by inhalation (e.g. aerosol inhalation). In another embodiment, the viral vector is supplied to the subject's respiratory mucosa.

Therapeutically effective virus amounts include but are not limited to 1×10⁷ viral particles (VPs), 5×10⁷ VPs, 1×10⁸ VPs, 5×10⁸ VPs, 1×10⁹ VPs, 5×10⁹ VPs, 1×10¹⁰ VPs, or more than 1×10¹⁰ VPs. Adenoviral vectors are particularly suitable for this purpose because of the large cargo capacity of the adenovirus. Suitable adenoviral vectors include those disclosed in WO 98/17783, WO 02/27007, WO 09/6479, & WO 14/31178, each of which is incorporated herein by reference in its entirety. Suitable methods for administering these adenoviral vectors are disclosed in WO 16/112188, which is herein incorporated by reference in its entirety.

In some embodiments, NAI be administered as a co-therapy along with an anti-SLAMF7 Fab fragment, antibody, biparatopic antibody, or CAR (or a pharmaceutical composition comprising the same). NAI may be administered separately in a separate composition or together in the same composition as the SLAMF7 Fab fragment, antibody, biparatopic antibody, or CAR (or a pharmaceutical composition comprising the same). NAI may be administered at a dose of about 5 μg/kg/day to about 15 μg/kg/day. In a particular embodiment, the NAI is administered at a dose of about 5 μg/kg/day, about 6 μg/kg/day, about 7 μg/kg/day, about 8 μg/kg/day, about 9 μg/kg/day, about 10 μg/kg/day, about 11 μg/kg/day, about 12 μg/kg/day, about 13 μg/kg/day, about 14 μg/kg/day, or about 15 μg/kg/day. In a specific embodiment, the NAI may be administered at about 10 μg/kg/day.

### EXAMPLES

The following example is provided to further illustrate the invention disclosed herein but, of course, should not be construed as in any way limiting its scope.

Example 1: Antigen-dependent cellular cytotoxicity (ADCC): Antibody dilution—Anti-SLAMF7 antibodies were diluted in Roswell Park Memorial Institute (RPMI) media at a stock concentration of 5 μg/mL (e.g., five times (5×) higher than the concentration used in the assay). 50 μL of the stock concentration were added to 200 μL of media in each well for a final volume of 250 μL and a concentration of 1 μg/mL.

Effector Preparation—NK92 cells (a high affinity NK (haNK) cell) were used as an effector cell. The NK92 cells were washed twice with complete media. After the last wash, the NK92 cells were resuspended wash with 2 mL of complete media and strain the cells were isolated with a 70 um strainer. The NK92 cells were diluted to a concentration of 2×10⁶ cells/mL in complete media. 100 μL of the NK92 effector cells were plated at the highest concentration. The effector cells were serially diluted five times at a 2:1 serial dilution.

Calcein acetoxymethyl (AM) staining of Target-Human embryonic kidney 293T (HEK293T) cells were used as target cells in the ADCC assay. The 293T cells were either unmodified or stably transfected to express the SLAMF7 peptide. The 293T target cells were centrifuged and the pellet was resuspended in 10 mL RPMI buffer and spiked with 10 μL of Calcein AM. The target cells were incubated with the Calcein AM for 20 minutes. After incubation with the Calcein AM, the stained target cells were washed twice in RPMI complete media. After the last wash, the target cells were resuspended with 10 mL of RPMI complete media and the cell density was calculated. The cells were further diluted to a concentration of 1×10⁵. 100 μL of the stained target cells were added to each well of the plate that contains the effector cells.

50 μL of the antibody dilution was added to each well. The plate was spun down and incubated for 3 hours. After 3 hours, a row of the target only cells was lysed and a sample of the supernatant from each sample was read via the plate reader, measuring Calcein AM release (excitation: 495 nm; emission: 515 nm).

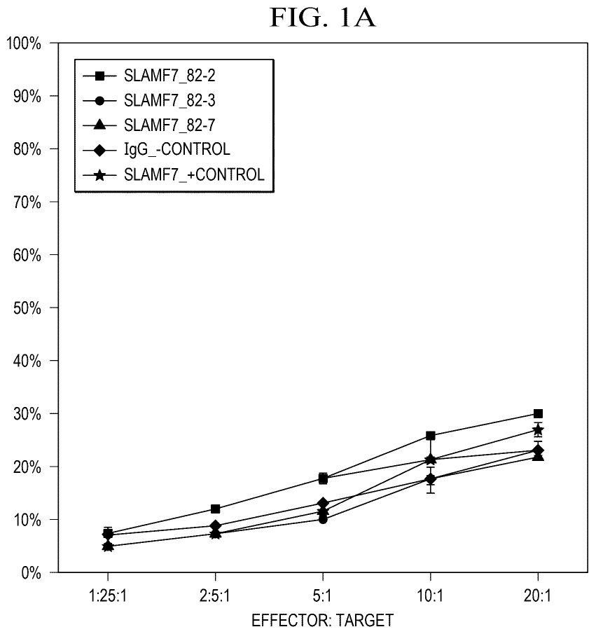

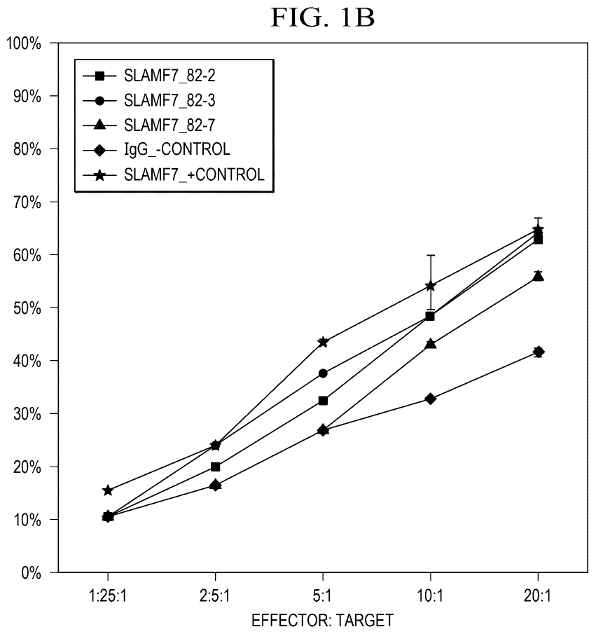

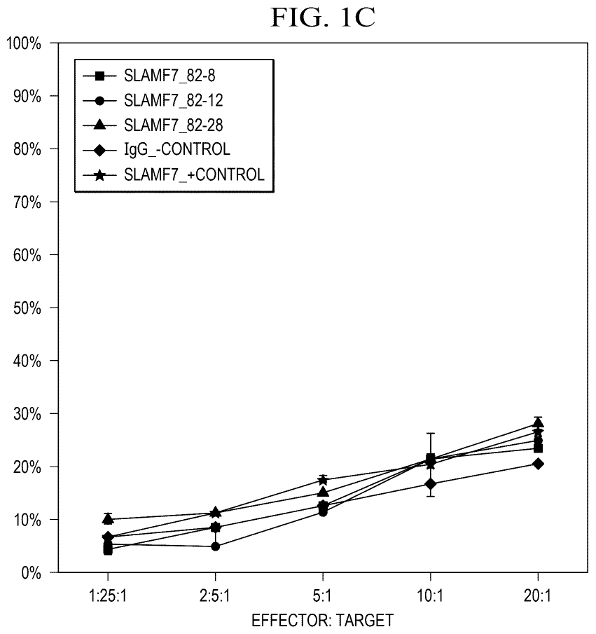

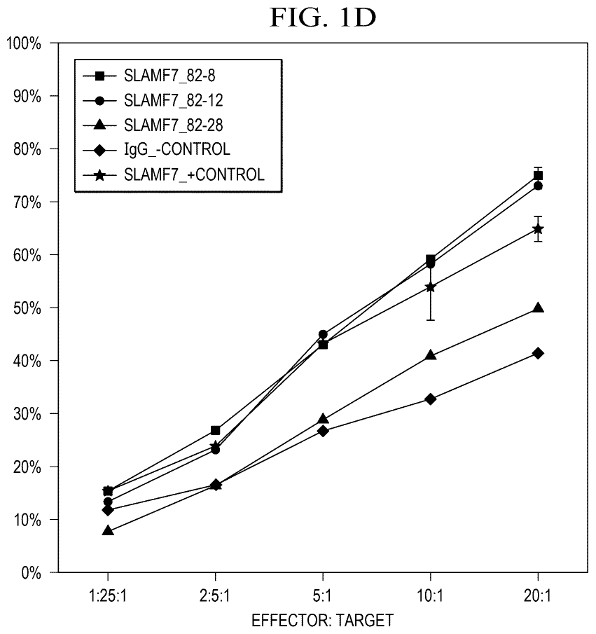

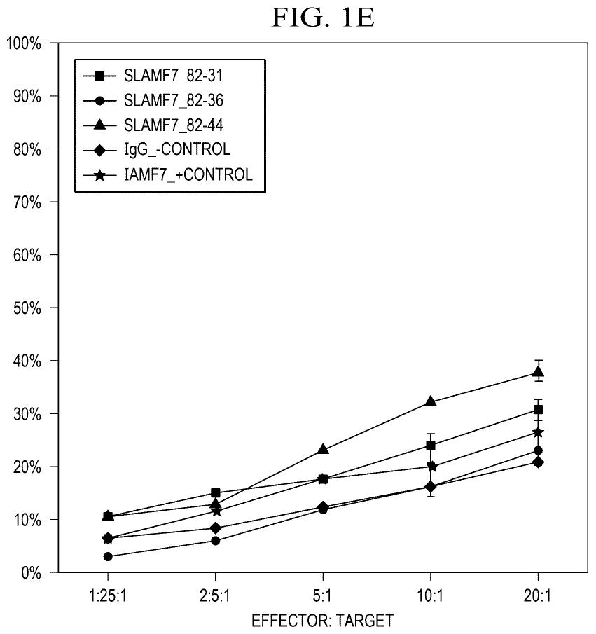

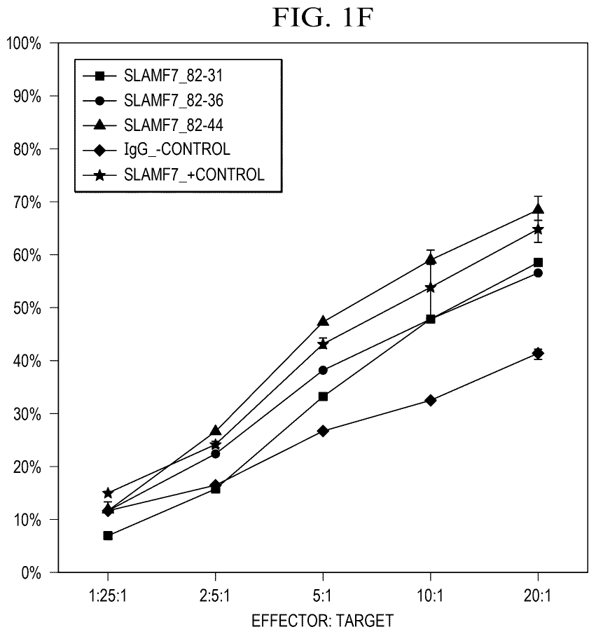

FIGS. 1A and 1B show the ADCC of species 82-2, 82-3, and 82-7 when the target is untransfected HEK 293T cells or HEK 293T cells stably expressing SLAMF7, respectively. FIGS. 1C and 1D show the ADCC of species 82-8, 82-12, and 82-28 when the target is untransfected HEK 293T cells or HEK 293T cells stably expressing SLAMF7, respectively. FIGS. 1E and 1F show the ADCC of species 82-31, 82-36, and 82-44 when the target is untransfected HEK 293T cells or HEK 293T cells stably expressing SLAMF7, respectively. In each experiment, the antibodies disclosed herein induce SLAMF7-specific ADCC in target cells expression SLAMF7 but ADCC does not occur or minimally occurs in target cells that do not express SLAMF7.

Example 2: ADCC EC₅₀ determination: Antibody dilution—Anti-SLAMF7 antibodies were diluted in RPMI at a stock concentration of 5,000 nM (e.g., five times (5×) higher than the concentration used in the assay). The antibody was then serially diluted at 15-fold dilutions to create additional stock concentrations of 5,000 nM, 500 nM, 50 nM, 5 nM, 0.5 nM, 0.05 nM, 0.005 nM, 0.0005 nM, and 0.00005 nM. 50 μL of each stock concentrations were added to 200 μL of media in each well for a final volume of 250 μL and a concentration of 1,000 nM, 200 nM, 40 nM, 8 nM, 1.6 nM, 0.32 nM, 0.064 nM, 0.0128 nM, 0.0026 nM, 0.0005 nM, 0.0001nM and 0.00002 nM. The control antibody is based on the sequence of HuLuc63 as described in U.S. Pat. No. 8,133,981, which is incorporated herein by reference.

NK92 cells were used as an effector cell. The NK92 cells were washed twice with complete media. After the last wash, the NK92 cells were resuspended wash with 2 mL of complete media and strain the cells were isolated with a 70 μm strainer. The NK92 cells were diluted to a concentration of 2×10⁶ cells/mL in complete media for an effector:target ratio of 20:1. 100 μL of the NK92 effector cells were plated.

Calcein AM staining of Target-HEK293T cells that express SLAMF7 were used as target cells in the ADCC EC₅₀ assay. The SLAMF7-expressing 293T target cells were centrifuged and the pellet was resuspended in 10 mL RPMI buffer and spiked with 10 μL of Calcein AM. The target cells were incubated with the Calcein AM for 20 minutes. After incubation with the Calcein AM, the stained target cells were washed twice in RPMI complete media. After the last wash, the target cells were resuspended with 10 mL of RPMI complete media and counted the cell density. The cells were further diluted to a concentration of 1×10⁵ cells/mL for an effector:target ratio of 20:1. 100 μL of the stained target cells were added to each well of the plate that contains the effector cells.

50 μL of the antibody dilutions were added to each well to create antibody concentrations ranging from 0.00001 nM to 1,000 nM at 5-fold increments. The plate was spun down and incubated for 4 hours. After 4 hours, a row of the target only cells was lysed and a sample of the supernatant from each sample was read via the plate reader, measuring Calcein release (excitation: 495 nm; emission: 515 nm).

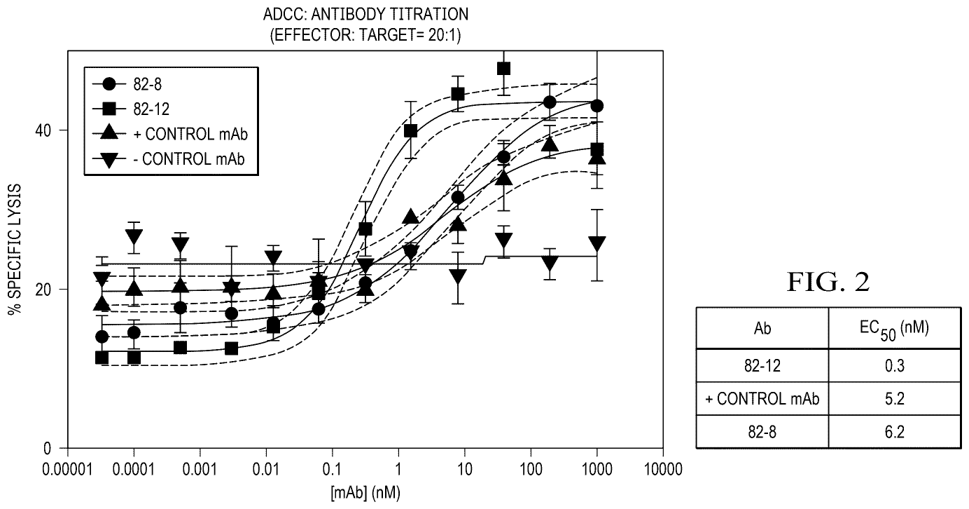

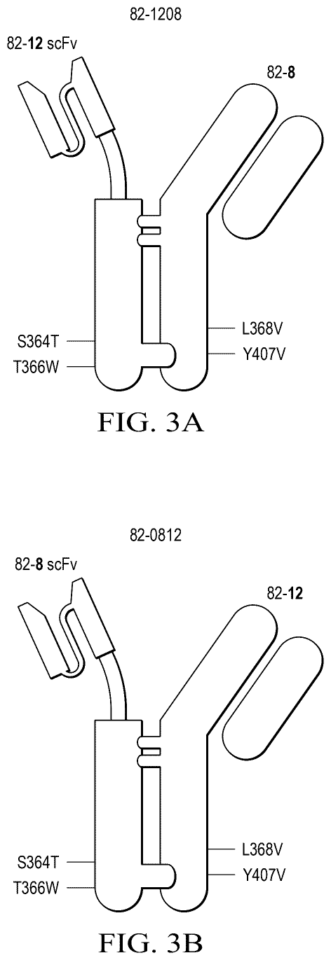

FIG. 2 shows the ADCC curve over the tested range of antibodies for species 82-8 and 82-12. The EC₅₀ for 82-12 is 0.3 nM and the EC₅₀ for 82-8 is 6.2 nM.

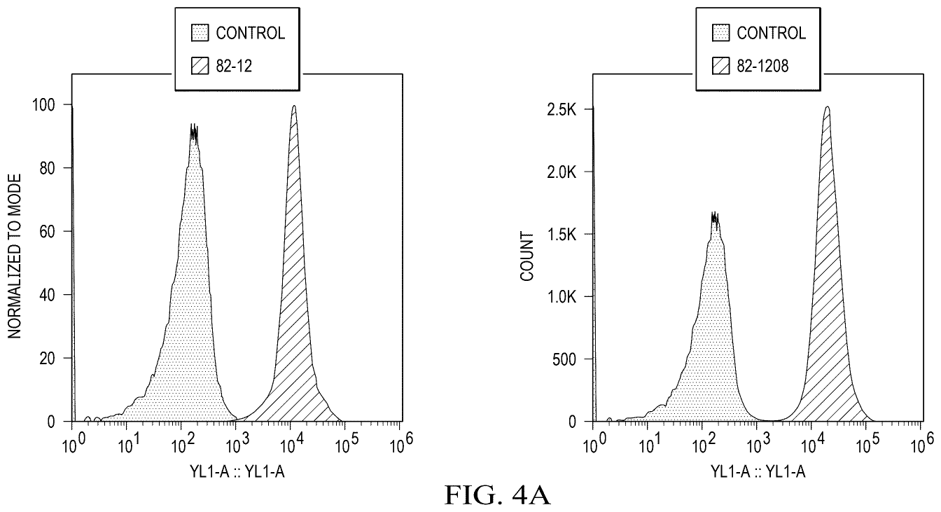

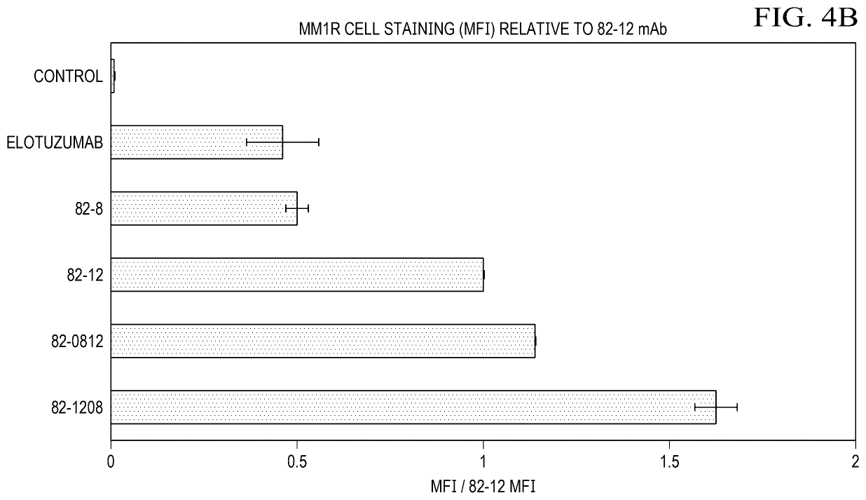

Example 3: Binding of biparatopic antibodies Biparatopic antibodies were also assayed for binding to SLAMF7. 200,000 MM1R cells, which express SLAMF7, were prepared in a similar manner as above. The cells were resuspended with 100 μL of cell separation buffer and a 1:100 dilution (10 μg/mL) of a control, non-specific antibody based on the HuLuc63 sequence disclosed above, 82-2 antibody, 82-12 antibody, biparatopic antibody 82-0812, or biparatopic antibody 82-1208. A 1:200 dilution of secondary anti-primary conjugated antibody was added to the cells and incubated for incubated at 4° C. for 30 minutes. After the incubation with the secondary antibody, the cells were washed twice with cell separation buffer and resuspended in 100 μL of cell separation buffer. Cell staining was measured by flow cytometry. FIG. 4A shows the staining of SLAMF7-expressing MM1R cells by control antibody, 82-12 antibody, or 82-1208 biparatopic antibody. FIG. 4B shows the relative cell staining of the tested antibodies and biparatopic antibodies as compared to mean fluorescent intensity (MFI) of the staining by 82-12.

Example 4: ADCC of biparatopic antibodies: Antibody dilution-Anti-SLAMF7 biparatopic antibodies were diluted in RPMI at a stock concentration of 5 μg/mL (e.g., five times (5×) higher than the concentration used in the assay). HuLuc63 (described above) was closed and used as a positive control. It is prepared as the biparatopic antibodies. 50 μL of the stock concentration were added to 200 μL of media in each well for a final volume of 250 μL and a concentration of 1 μg/mL.

Effector Preparation-NK92 cells expressing CD16 (haNK) were used as an effector cell. The NK92 cells were washed twice with complete media. After the last wash, the NK92 cells were resuspended wash with 2 mL of complete media and strain the cells were isolated with a 70 um strainer. The NK92 cells were diluted to a concentration of 2×10⁶ cells/mL in complete media. 100 μL of the NK92 effector cells were plated at the highest concentration. The effector cells were serially diluted five times at a 2:1 serial dilution.

Calcein AM staining of Target-MM1R cells were used as target cells in the ADCC assay. The MM1R target cells were centrifuged and the pellet was resuspended in 10 mL RPMI buffer and spiked with 10 μL of Calcein AM. The target cells were incubated with the Calcein AM for 20 minutes. After incubation with the Calcein AM, the stained target cells were washed twice in RPMI complete media. After the last wash, the target cells were resuspended with 10 mL of RPMI complete media and counted the cell density. The cells were further diluted to a concentration of 1×10⁵ cells/mL. 100 μL of the stained target cells were added to each well of the plate that contains the effector cells.

50 μL of the biparatopic antibody dilution was added to each well. The plate was spun down and incubated for 3 hours. After 3 hours, a row of the target only cells was lysed and a sample of the supernatant from each sample was read via the plate reader, measuring Calcein release (excitation: 495 nm; emission: 515 nm).

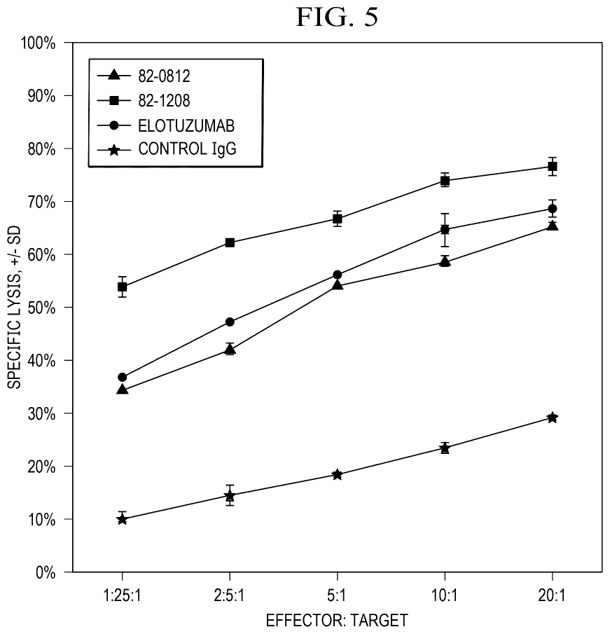

FIG. 5 shows the ADCC of elotuzumab and biparatopic antibodies 82-0812, and 82-1208. Both biparatopic antibodies induce as much or more ADCC of the target cells as compared to elotuzumab.

Example 5: Antibody-dependent cellular phagocytosis (ADCP) (antibody titration): 40 μg/mL stock of each antibody was made and serially diluted eight times at a 1:10 ratio in a 96-well plate. 1.5×10⁶ THP-1 cells (a human macrophage cell line) were spun down and washed twice with RPMI complete media. The cells were then resuspended in 5 mL and passed through a 40 μm filter. The cells were diluted to 2×10⁵ cells/mL.

Carboxyfluorescein succinimidyl ester (CFSE) staining of MM1R-10⁶ MM1R target cells were resuspended with 20 mL of phosphate buffered saline (PBS). 18 μL of DMSO-dissolved CFSE stock solution was added to the cells and incubated for 20 minutes at room temperature. After 20 min, the cells were pelleted and resuspended with 3 mL of fetal bovine serum (FBS) and incubated at 37° C. in a water bath for 5 minutes. The cells were pelleted and twice washed with RPMI complete. After the last wash, the cells were resuspended with 5 mL of complete RPMI media. The cells were then filtered through a 40 μm filter and were counted. The cells were diluted to a final 5:1 effector (E):target (T) ratio with complete RPMI. 50 μL of the target cell suspension was added to each well with the effector and antibody.

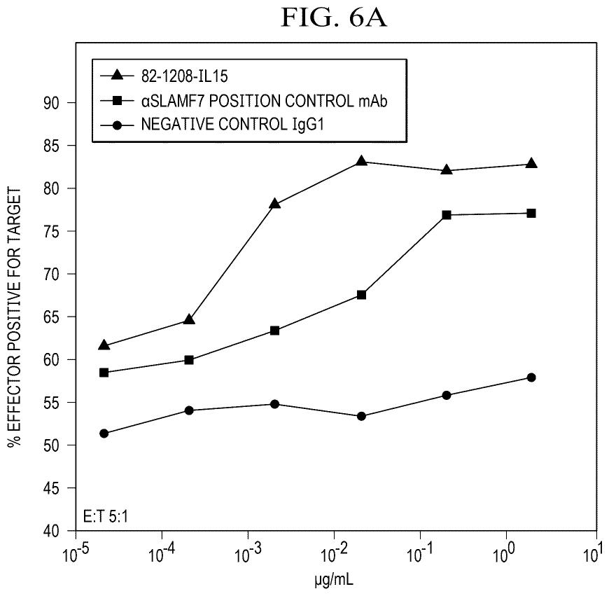

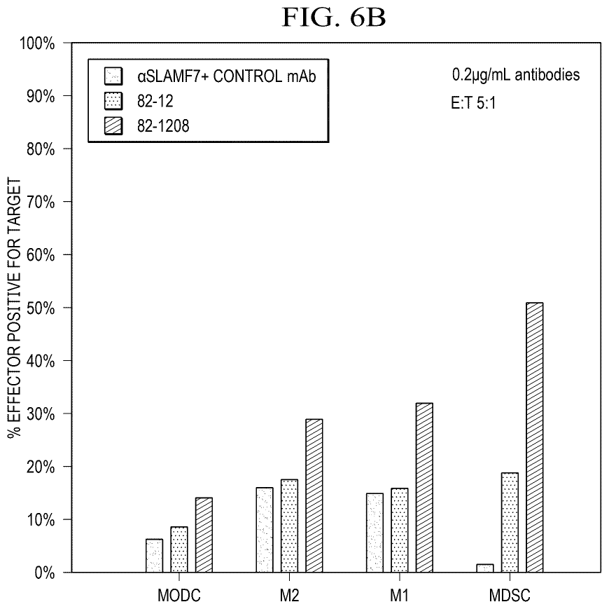

Reading the ADCP—After 2 hours in culture, the plate was spun down. The cells were washed twice in azide-free and serum/protein-free PBS. The supernatant was completely decanted. 120 μL of HLA-A2 APC (eBioscience) was added to 25 mL of PBS without calcium or magnesium. 25 μL of fixable viability stain 780 (FVD 780) (eBioscience) was added to the HLA-A2 APC-PBS mixture and 100 μL of each antibody cocktail was added to each well. The plate was incubated for 30 minutes at room temperature and protected from light. Following incubation, the cells were washed twice with cell separation buffer (comprising PBS, EDTA, and BSA). The cells were then assayed via flow cytometry and gating on the live (FVD negative) population and the double positive (HLA-A2-THP-1 and CFSE positive) cells. FIG. 6A shows the ADCP of the MM1R target cells from THP1 effector cells following incubation with increasing concentrations of biparatopic antibody 82-1208-IL15.

Example 6: Antibody-dependent cellular phagocytosis (ADCP) (fixed antibody concentration): Macrophage Preparation-THP-1 cells and primary macrophages were utilized in this experiment. Whole blood was obtained from donors and subjected to a FICOLL gradient to remove red blood cells and isolate peripheral blood mononuclear cells (PBMCs). PBMCs were cultured in RPMI (1% FBS) at a concentration of 3-5×10⁶ cells/mL for 1-2 hrs at 37° C. Non-adherent cells were removed. The culture flasks were rinsed three times with RPMI devoid of FBS. The enriched PMBCs were then cultured at 37° C. for 6 days in RPMI (10% FBS) containing a macrophage growth factor (M-CSF or GM-CSF). A population of cells were incubated with M-CSF (50 ng/ml) alone (M2) and further treated as below. Another population of cells were incubated with M-CSF (50 ng/mL) and GM-CSF (25 ng/mL) (M1) and further treated as below. The M1 and M2 cells were then differentiated into macrophage subpopulations.

The culture from the supernatant was removed and pelleted to recover any cells that detached during culture. The recovered cells were added back to the culture dish containing adhered cells. The cells were cultured in RPMI (10% FBS) media with the M-CSF, GM-CSF, IFN-γ (50 ng/mL) and LPS (100 ng/mL) (for M2) or M-CSF and IL-4 (20 ng/ml) (for M2) for 24 hr at 37° C. Non-adherent cells were collected and pelleted in a 15 mL conical tube. The adherent cells in the culture flask were rinsed with PBS (1-2 mM EDTA) and then incubated in PBS (1-2 mM EDTA) at 37° C. for five to ten minutes. The cells were removed from the flask via vigorous pipetting. The dislodged cells were pelleted and counted. The cells were assayed for CD206 (mannose receptor), CD86 (B7.2), and CD197 (CCR7) by flow cytometry. M1 cells express higher levels of CD86 and CD197 relative to M2. M2 cells express higher levels of CD206 relative to M1.

THP-1 or the primary macrophages were labelled in suspension. CFSE (CellTrace) stock solution was prepared immediately prior to use by adding the appropriate volume of DMSO (20 μL). 1 μL CFSE stock solution was then added to each mL of cell suspension in PBS for a final working solution. Macrophages were incubated with the CFSE and for 20 minutes at 37° C., protected from light. Five times the original staining volume of culture medium (containing at least 1% protein) was added to the cells and incubated for 5 minutes to remove any free dye remaining in the solution. The macrophages were pelleted by centrifugation and resuspended in 2.5 mL of fresh, pre-warmed complete culture medium. The macrophages were incubated for at least 10 minutes before analysis to allow the CFSE reagent to undergo acetate hydrolysis. The macrophages were counted for viability and concentration. Additional media was added to make the concentration of cells to 56,000 cells/mL for a 5:1 effector to target ratio. 50 μL of the cells were plated over the entire plate of the antibody titrations.

Preparation of the antibody in media—0.4 μg/mL dilution of every antibody was prepared by combining 5 mL of media with 1 μg of antibody. 100 μL of each dilution was distributed to each well of a 96-well plate.

CFSE staining of MM1R-10⁶ MM1R target cells were resuspended with 20 mL of PBS. 18 μL of CFSE stock solution was added to the cells and incubated for 20 minutes at room temperature. After 20 min, the cells were pelleted and resuspended with 3 mL of fetal bovine serum (FBS) and incubated at 37° C. in a water bath for 5 minutes. The cells were pelleted and twice washed with RPMI complete. After the last wash, the cells were resuspended with 5 mL of complete RPMI media. The cells were then filtered through a 40 μm filter and were counted. The cells were diluted to a final concentration of 11,000 cells/mL with complete RPMI.

Reading the ADCP—After 2 hours in culture, the plate was spun down. The cells were washed twice in azide-free and serum/protein-free PBS. The supernatant was completely decanted. 30 mL of azide-and serum/protein-free PBS without calcium and magnesium was combined with 30 mL of PBS without calcium or magnesium was combined with 30 microliters of fixable viability stain 780 (FVD 780) (eBioscience) and 100 μL of each antibody cocktail was added to each well and incubated for 30 minutes at room temperature and protected from light. Following incubation, the cells were washed once or twice with cell separation buffer. The cells were then assayed via flow cytometry and gating on the live population and the double positive and CFSE positive cells.

FIG. 6B shows the ADCP activity of primary monocyte derived dendritic cells, M1 and M2 macrophages, and myeloid derived suppressor cells against MM1R target cells in the presence of anti-SLAMF7 positive control antibody, biparatopic antibody 82-12, and biparatopic antibody 82-1208.

Example 7: ADCP+overnight stimulation with N-803 using primary NK cells targeting MM1R.

The antigen-dependent cellular phagocytosis of primary NK cells was determined. The protocol was the same as described in Example 5 above except that primary NK cells and macrophages were used instead of THP-1 cells.

Primary NK cells were prepared with the NK Cell Isolation kit (Miltenyi Biotec) according to manufacturer instructions. Briefly, whole blood was obtained from donors and subjected to a Ficoll gradient to remove red blood cells and isolate PBMCs. The PBMCs were resuspended in a PBS buffer (0.5% BSA, 2 mM EDTA, pH 7.2) at a concentration of 10⁷ cells per 40 μL. 10 μL of the NK Cell Biotin-Antibody cocktail were added to each to the resuspended cells for each 10⁷ cells (40 μL buffer) and incubated at 2-8° C. for 5 minutes. The cells were then diluted with an additional 30 μL PBS buffer and 20 μL NK Cell MicroBead Cocktail per each 10⁷ cells (40 μL buffer). The cells were then incubated at 2-8° C. for 10 minutes.

The Magnetic-activated cell sorting (MACS) column was prepared by attaching the column to a magnetic separator and rinsing the column with PBS buffer. The cell suspension was applied to the MACS column. Unlabeled, non-NK cells pass through the column and can be collected. The MACS column was washed with the PBS buffer to remove any cells physically, but not magnetically, retained in the column. The MACS column was then removed from the separator and the magnetically retained cells (e.g., NK cells) were collected. The MACS column was washed with PBS buffer to flush out remaining cells.

Immature and mature monocyte derived dendritic cells (MDDCs) were prepared from whole blood. Whole blood was subjected to a Ficoll gradient. The cells were washed twice with RPMI and pelleted at following centrifugation at 1000 rpm. The supernatant was aspirated to remove platelets. To obtain CD83⁻ (immature) monocyte-derived dendritic cells, the monocytes were cultured at 3-5×10⁶ cells/mL for 2 hrs at 37° C. (1% FCS). Non-adherent cells were removed and the flask was rinsed three times with RPMI. Adherent cells were then cultured for 3 days in RPMI (10% FCS) with IL-4 (100 U/mL) and GM-CSF (200 U/mL) for three days. Media with detached cells are transferred to 50 mL conical tubes. The flask was rinsed with RPMI (pH 7.4; warmed to 37° C.). The media from the rinsed flask were combined with the detached cells. 5 mL PBS (1 mM EDTA) was added to the flask and incubated for 10 minutes. The flask was rapped to detach adherent cells. The detached cells were added to the decanted cells/media. The cells were washed twice with RPMI (10% FCS) and CD1a, CD1b, and CD1c expression was determined by flow cytometry.

To obtain CD83⁺ (mature) monocyte-derived dendritic cells, the monocytes were cultured at 8×10⁶ cells/mL for 2 hrs at 37° C. (10% FCS). Non-adherent cells were removed and the flask was rinsed three times with RPMI. Adherent cells were then cultured for 3 days in RPMI (10% FCS) with IL-4 (500 U/mL) and GM-CSF (800 U/mL) for five days. TNF-α (100 U/mL) was added to the incubation media and the cells were cultured for an additional 2 days. Media with detached cells are transferred to 50 mL conical tubes. The flask was rinsed with RPMI (pH 7.4; warmed to 37° C.). The media from the rinsed flask were combined with the detached cells. 5 mL PBS (1 mM EDTA) was added to the flask and incubated for 10 minutes. The flask was rapped to detach adherent cells. The detached cells were added to the decanted cells/media. The cells were washed twice with RPMI (10% FCS) and CD1a, CD1b, and CD1c expression was determined by flow cytometry.

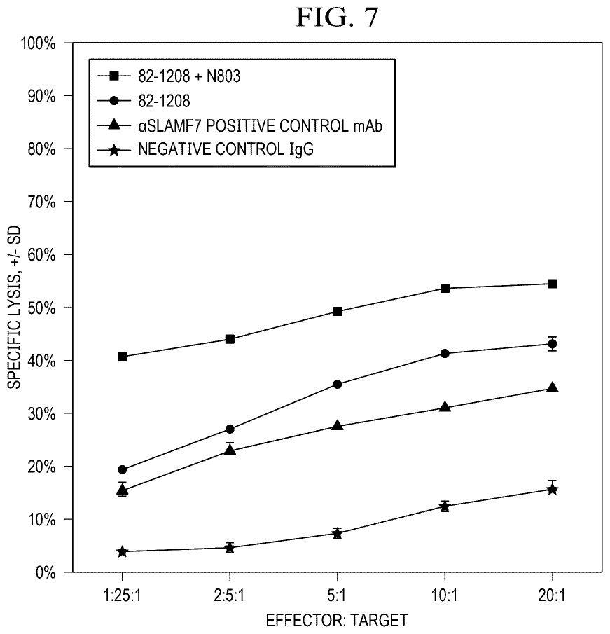

FIG. 7 shows the ADCC activity of the primary NK cells against MM1R target cells in the presence of an anti-SLAMF7 positive control antibody, biparatopic antibody 82-1208, or biparatopic antibody in the presence of N-803 (an IL-15 agonist).

### Example 8: αSLAMF7 Bispecific-IL15 Fusion ADCP Activity

The antigen-dependent cellular phagocytosis of primary NK cells was determined. The protocol was the same as described in Example 5 above except that anti-SLAMF7 bispecific IL-15 fusion antibodies (82-1208-IL-15) and 82-12-IL-15) are used instead.

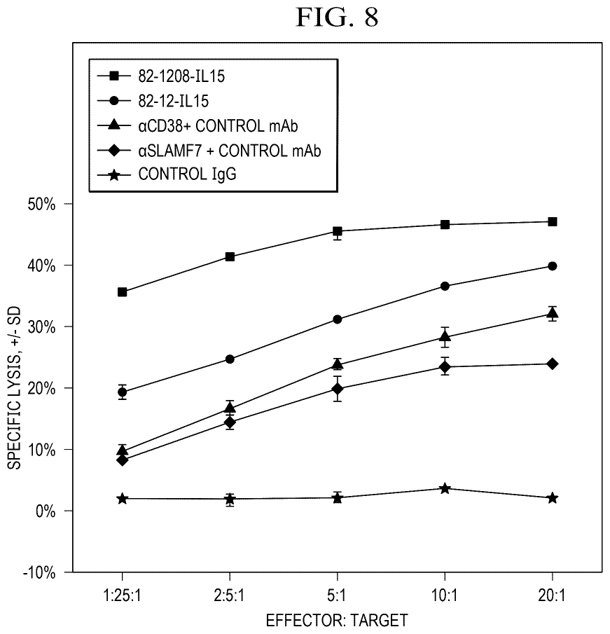

FIG. 8 shows the ADCC of 82-1208-IL15 and 82-12-IL15. Both fusion peptides antibodies induce more ADCC of the target cells as compared to anti-CD38 and control mAb or anti-SLAMF7 and control mAb.

Example 9: CAR-dependent cellular cytotoxicity (CAR-DCC): CAR production—Primary T cells frozen in complete media were thawed, counted, and pelleted. The T cells were resuspended in 10 mL electroporation buffer (RPMI media) and spun down (400 rpm for 15 minutes). The pellet was resuspended in electroporation buffer. mRNA encoding 82-12 CAR, 82-8 CAR, or control were added to the resuspended T cells. The cuvette was shocked (conditions) and rested. After electroporation the cells were transferred to a culture flask with complete media and cultured for 1 hr at 37° C. The electroporated T cells were used as effector cells.

Effector cell preparation—Effector cells were washed twice with complete media. The cells were resuspended with 2 mL of complete media and strained with a 70 μm strainer. The effector cells were diluted to the desired concentration with complete media. 100 μL of the effector cells were plated at the highest concentration and serially diluted with 2:1 serial dilutions.

Calcein AM staining of target cells—Target cells (either HEK 293T cell stably expressing SLAMF7 or MM1R cells) were pelleted and resuspended with 10 mL RPMI and spiked with 10 μL of calcein AM. The target cells were stained for 20 minutes. The cells were then spun down and washed twice with RPMI complete media. The cells were then resuspended with 10 mL of complete media and counted. The cells were diluted to the desired concentration to an appropriate multiple of the effector cells. 100 μL of the target cells were added to each well. The plate was then spun down and incubated for 4 hours. After 4 hours, a row of the target only cells was lysed and a sample of the sample of the supernatant was read via a plate reader to measure calcein AM release (excitation: 495 nm; emission: 515 nm).

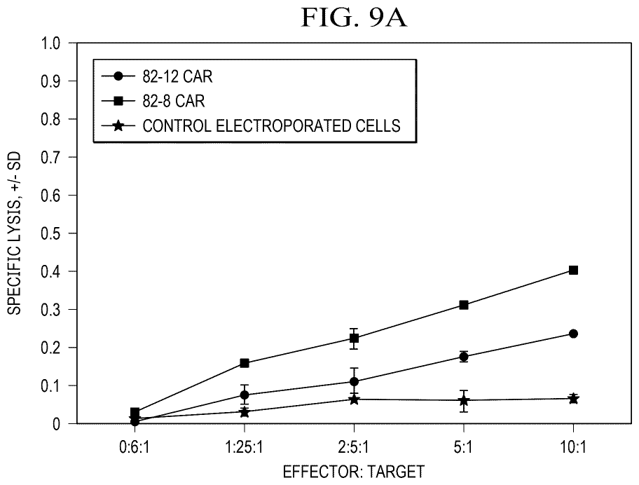

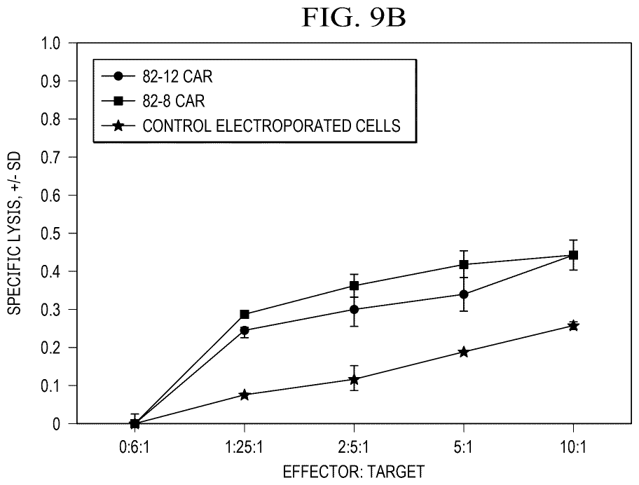

FIG. 9A shows the CAR-DCC of 82-12 CAR and 82-8 CAR with HEK 293T that stably express SLAMF7 as the target cells. FIG. 9B shows the CAR-DCC of 82-12 CAR and 82-8 CAR with MM1R as the target cells.

Example 10: CAR-DCC with ADCC: Primary T cells were prepared and electroporated as in Example 8, with the exception that only 82-8 CAR is used. Biparatopic antibody 82-1208 was prepared as in Example 4. 50 μL of the stock concentration were added to 200 μL of media in each well for a final volume of 250 μL and a concentration of 1 μg/mL.

Effector cell preparation—Effector haNK cells were washed twice with complete media. The cells were resuspended with 2 mL of complete media and strained with a 70 μm strainer. The effector cells were diluted to the desired concentration with complete media. 100 μL of the effector cells were plated at the highest concentration and serially diluted with 2:1 serial dilutions.

Calcein AM staining of target cells—MM1R target cells were pelleted and resuspended with 10 mL RPMI and spiked with 10 μL of calcein AM. The target cells were stained for 20 minutes. The cells were then spun down and washed twice with RPMI complete media. The cells were then resuspended with 10 mL of complete media and counted. The cells were diluted to the desired concentration to an appropriate multiple of the effector cells.

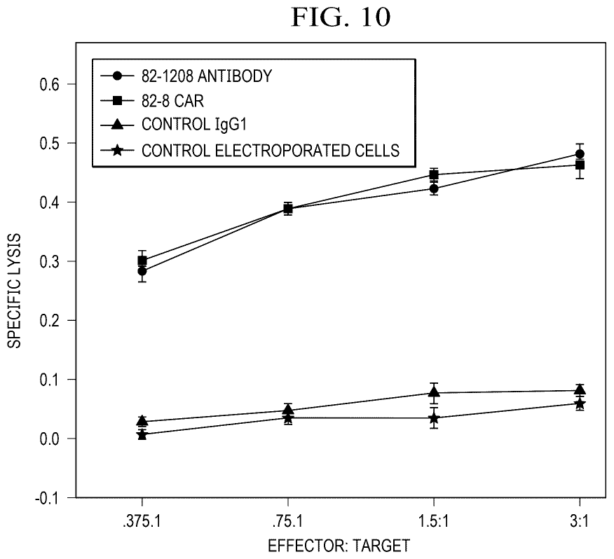

100 μL of the target cells were added to each well. The plate was then spun down and incubated for 4 hours. 50 μL of the biparatopic 82-1208 was added to the wells with haNK effector cells. the 82-1208 was not added to wells containing the primary T cells electroporated with 82-8 CAR mRNA. After 4 hours, a row of the target only cells was lysed and a sample of the supernatant was read via a plate reader to measure calcein AM release (excitation: 495 nm; emission: 515 nm). FIG. 10 shows that both 82-1208 biparatopic antibody and 82-8 CAR induce cellular cytotoxicity.

Example 11: SLAM Ab Cross-Reactivity Determination. SLAM family members antibody cross-reactivity can be determined through standard methods known in the art. For example, heterologous expression of SLAM receptors in CHO or HEK cells, along with binding assays or cell signaling assays can be used to determine activities against non-SLAMF7 members of the SLAM family.

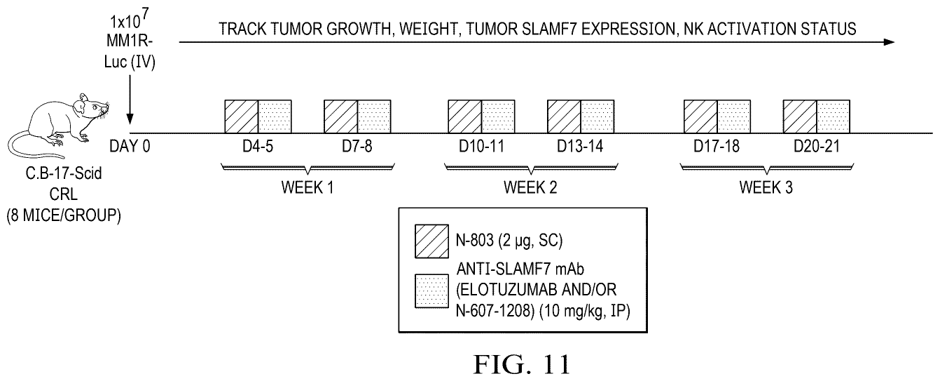

Example 12: N-803 and N-607-1208 (82-LH1208) combination therapy in disseminated MM1R multiple myeloma model. Six groups of C.B-17-Scid mice (8 mice per group) are administered 1×10⁷ MM1R-Luc B lymphoblast cells via intravenous (IV) route on day zero. The tumor growth, weight, tumor SLAMF7 expression, and NK activation status are tracked through weeks 1, 2 and 3 according to FIG. 11. The six groups of mice are given the following treatments shown in Table 12.

TABLE 12

| Group # | Mouse Strain (n) | Treatment/mouse | Route |
|---|---|---|---|
| 1 | C.B-17-Scid (8) | phosphate buffered | Intraperitoneal (IP) |
| | | saline (PBS) | |
| 2 | C.B-17-Scid (8) | Elotuzumab | IP |
| | | (10 mg/kg) | |
| 3 | C.B-17-Scid (8) | N-607-1208 | IP |
| | | (10 mg/kg) | |
| 4 | C.B-17-Scid (8) | N-803 | Subcutaneous (SC) |
| | | (2 μg) | |
| 5 | C.B-17-Scid (8) | Elotuzumab | IP |
| | | (10 mg/kg) | |
| | | N-803 | SC |
| | | (2 μg) | |
| 6 | C.B-17-Scid (8) | N-607-1208 | IP |
| | | (10 mg/kg) | |
| | | N-803 | SC |
| | | (2 μg) | |

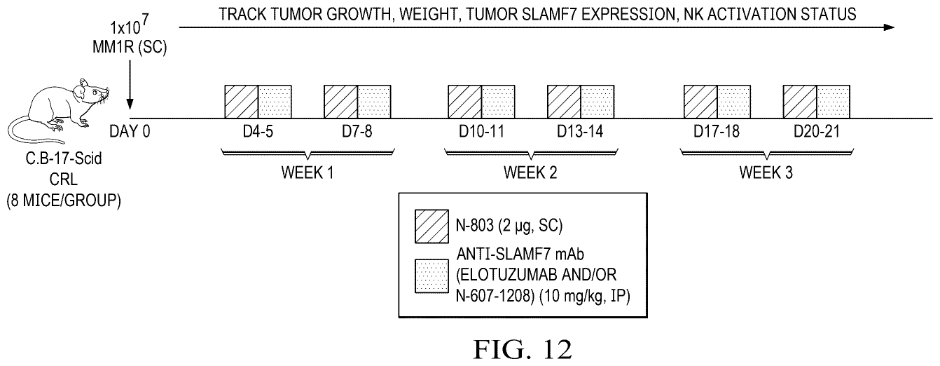

Example 13: N-803 and N-607-1208 (82-LH1208) combination therapy in subcutaneous MM1R multiple myeloma model. Six groups of C.B-17-Scid mice (8 mice per group) are administered 1×10⁷ MM1R-Luc B lymphoblast cells via subcutaneous (SC) route on day zero. The tumor growth, weight, tumor SLAMF7 expression, and NK activation status are tracked through weeks 1, 2 and 3 according to FIG. 12. The six groups of mice are given the following treatments shown in Table 13.

TABLE 13

| Group # | Mouse Strain (n) | Treatment/mouse | Route |
|---|---|---|---|
| 1 | C.B-17-Scid (8) | phosphate buffered | Intraperitoneal (IP) |
| | | saline (PBS) | |
| 2 | C.B-17-Scid (8) | Elotuzumab | IP |
| | | (10 mg/kg) | |
| 3 | C.B-17-Scid (8) | N-607-1208 | IP |
| | | (10 mg/kg) | |
| 4 | C.B-17-Scid (8) | N-803 | Subcutaneous (SC) |
| | | (2 μg) | |
| 5 | C.B-17-Scid (8) | Elotuzumab | IP |
| | | (10 mg/kg) | |
| | | N-803 | SC |
| | | (2 μg) | |
| 6 | C.B-17-Scid (8) | N-607-1208 | IP |
| | | (10 mg/kg) | |
| | | N-803 | SC |
| | | (2 μg) | |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Particular embodiments of this invention are described herein, including the best mode known to the inventors for carrying out the invention. Variations of those particular embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A biparatopic antibody comprising a first arm and a second arm, wherein:
the first arm comprises a first heavy variable (V_{H}) chain having at least 85% sequence identity to each of SEQ ID NO: 37, 38, 39, and 40 and a first light variable (V_{L}) chain having at least 85% identity to each of SEQ ID NOs: 41, 42, 43, and 44,
wherein the first V_{H} chain and the first V_{L} chain are linked via a linker, and
the second arm comprises a second V_{H} chain having at least 85% sequence identity to each of SEQ ID NO: 37, 38, 39, and 40 and a second V_{L} chain having at least 85% identity to each of SEQ ID NOs: 41, 42, 43, and 44,
wherein each of the first V_{H} chain and the first V_{L} chain have three complementarity determining regions (CDRs), wherein:
the first V_{H} chain comprises SEQ ID NOs: 1, 2, and 3 and the first V_{L} chain comprises SEQ ID NOs: 25, 26, and 27;
the first V_{H} chain comprises SEQ ID NOs: 4, 5, and 6and the first V_{L} chain comprises SEQ ID NOs: 25, 26, and 28;
the first V_{H} chain comprises SEQ ID NOs: 7, 8, and 9and the first V_{L} chain comprises SEQ ID NOs: 25, 26, and 29;
the first V_{H} chain comprises SEQ ID NOs: 4, 10, and 11 and the first V_{L} chain comprises SEQ ID NOs: 25, 26, and 30;
the first V_{H} chain comprises SEQ ID NOs: 12, 5, and 13and the first V_{L} chain comprises SEQ ID NOs: 25, 26, and 31;
the first V_{H} chain comprises SEQ ID NOs: 4, 14, and 15and the first V_{L} chain comprises SEQ ID NOs: 25, 26, and 32;
the first V_{H} chain comprises SEQ ID NOs: 7, 16, and 17and the first V_{L} chain comprises SEQ ID NOs: 25, 26, and 33;
the first V_{H} chain comprises SEQ ID NOs: 18, 19, and 20and the first V_{L} chain comprises SEQ ID NOs: 25, 91, and 34;
the first V_{H} chain comprises SEQ ID NOs: 4, 21, and 22 and the first V_{L} chain comprises SEQ ID NOs: 25, 26, and 35; or.
the first V_{H} chain comprises SEQ ID NOs: 4, 23, and 24 and the first V_{L} chain comprises SEQ ID NOs: 25, 26, and 36,
wherein each of the second V_{H} chain and the second V_{L} chain have three complementarity determining regions (CDRs), wherein:
the second V_{H} chain comprises SEQ ID NOs: 1, 2, and 3 and the second V_{L} chain comprises SEQ ID NOs: 25, 26, and 274;
the second V_{H} chain comprises SEQ ID NOs: 4, 5, and 6 and the second V_{L} chain comprises SEQ ID NOs: 25, 26, and 28;
the second V_{H} chain comprises SEQ ID NOs: 7, 8, and 9 and the second V_{L} chain comprises SEQ ID NOs: 25, 26, and 29;
the second V_{H} chain comprises SEQ ID NOs: 4, 10, and 11 and the second V_{L} chain comprises SEQ ID NOs: 25, 26, and 30;
the second V_{H} chain comprises SEQ ID NOs: 12, 5, and 13 and the second V_{L} chain comprises SEQ ID NOs: 25, 26, and 31;
the second V_{H} chain comprises SEQ ID NOs: 4, 14, and 15 and the second V_{L} chain comprises SEQ ID NOs: 25, 26, and 32;
the second V_{H} chain comprises SEQ ID NOs: 7, 16, and 17 and the second V_{L} chain comprises SEQ ID NOs: 25, 26, and 33;
the second V_{H} chain comprises SEQ ID NOs: 18, 19, and 20 and the second V_{L} chain comprises SEQ ID NOs: 25, 91, and 34;
the second V_{H} chain comprises SEQ ID NOs: 4, 21, and 22 and the second V_{L} chain comprises SEQ ID NOs: 25, 26, and 35; or.
the second V_{H} chain comprises SEQ ID NOs: 4, 23, and 24 and the second V_{L} chain comprises SEQ ID NOs: 25, 26, and 36, and.
wherein the first arm and the second arm comprise different V_{H} and V_{L} chains.

2. The biparatopic antibody of claim 1, wherein the first arm further comprises a crystallizable fragment (Fc) domain.

3. The biparatopic antibody of claim 2, wherein the first arm has an arrangement of first V_{H} chain-linker-first V_{L} chain-linker-Fc.

4. The biparatopic antibody of claim 3, wherein:
the first arm has an arrangement of (SEQ ID NO: 37)-V_{H} CDR1-(SEQ ID NO: 38)-V_{H} CDR2-(SEQ ID NO: 39)-V_{H} CDR3-(SEQ ID NO: 40)-linker-(SEQ ID NO: 41)-V_{L} CDR1-(SEQ ID NO: 38)-V_{L} CDR2-(SEQ ID NO: 43)-V_{L} CDR3-(SEQ ID NO: 44)-linker-Fc.

5. The biparatopic antibody of claim 4, wherein:
the first arm has an arrangement of (SEQ ID NO: 37)-(SEQ ID NO: 12)-(SEQ ID NO: 38)-(SEQ ID NO: 5)-(SEQ ID NO: 39)-(SEQ ID NO: 13)-(SEQ ID NO: 40)-linker-(SEQ ID NO: 41)-(SEQ ID NO: 25)-(SEQ ID NO: 42)-(SEQ ID NO: 26)-(SEQ ID NO: 43)-(SEQ ID NO: 31)-(SEQ ID NO: 44)-linker-(SEQ ID NO: 46), and
in the second arm, the V_{L} chain has an arrangement of (SEQ ID NO: 41)-(SEQ ID NO: 25)-(SEQ ID NO: 42)-(SEQ ID NO: 26)-(SEQ ID NO: 43)-(SEQ ID NO: 30)-(SEQ ID NO: 44) and the V_{H} chain has an arrangement of (SEQ ID NO: 37)-(SEQ ID NO: 4)-(SEQ ID NO: 38)-(SEQ ID NO: 10)-(SEQ ID NO: 39)-(SEQ ID NO: 11)-(SEQ ID NO: 40-(SEQ ID NO: 45)-(SEQ ID NO: 46).

6. The biparatopic antibody of claim 4, wherein:
the first arm has an arrangement of (SEQ ID NO: 37)-(SEQ ID NO: 4)-(SEQ ID NO: 38-(SEQ ID NO: 10)-(SEQ ID NO: 39)-(SEQ ID NO: 11)-(SEQ ID NO: 40)-linker-(SEQ ID NO: 41)-(SEQ ID NO: 25)-(SEQ ID NO: 42)-(SEQ ID NO: 26)-(SEQ ID NO: 43-(SEQ ID NO: 30)-(SEQ ID NO: 44)-linker-(SEQ ID NO: 46), and
in the second arm, the V_{L} chain has an arrangement of (SEQ ID NO: 41)-(SEQ ID NO: 25-(SEQ ID NO: 42)-(SEQ ID NO: 26)-(SEQ ID NO: 43)-(SEQ ID NO: 31)-(SEQ ID NO: 44) and the V_{H} chain has an arrangement of (SEQ ID NO: 37)-(SEQ ID NO: 12)-(SEQ ID NO: 38)-(SEQ ID NO: 5)-(SEQ ID NO: 39)-(SEQ ID NO: 13)-(SEQ ID NO: 40-(SEQ ID NO: 45)-(SEQ ID NO: 46).

7. The biparatopic antibody of claim 2, wherein the first arm has an arrangement of first V_{L} chain-linker-first V_{H} chain-linker-Fc.

8. The biparatopic antibody of claim 7, wherein:
the first arm has an arrangement of (SEQ ID NO: 41)-V_{L} CDR1-(SEQ ID NO: 42)-V_{L} CDR2-(SEQ ID NO: 43)-V_{L} CDR3-(SEQ ID NO: 44)-linker-(SEQ ID NO: 37)-V_{H} CDR1-(SEQ ID NO: 38)-V_{H} CDR2-(SEQ ID NO: 39)-V_{H} CDR3-(SEQ ID NO: 40-linker-Fc.

9. The biparatopic antibody of claim 8, wherein:
the first arm has an arrangement of (SEQ ID NO: 41)-(SEQ ID NO: 25)-(SEQ ID NO: 42-(SEQ ID NO: 26)-(SEQ ID NO: 43)-(SEQ ID NO: 31)-(SEQ ID NO: 44)-linker-(SEQ ID NO: 37)-(SEQ ID NO: 126)-(SEQ ID NO: 38)-(SEQ ID NO: 5)-(SEQ ID NO: 39-(SEQ ID NO: 13)-(SEQ ID NO: 40)-linker-(SEQ ID NO: 46), and
in the second arm, the V_{L} chain has an arrangement of (SEQ ID NO: 41)-(SEQ ID NO: 25-(SEQ ID NO: 42)-(SEQ ID NO: 26)-(SEQ ID NO: 43)-(SEQ ID NO: 33)-(SEQ ID NO: 44) and the V_{H} chain has an arrangement of (SEQ ID NO: 37)-(SEQ ID NO: 4)-(SEQ ID NO: 38)-(SEQ ID NO: 10)-(SEQ ID NO: 39)-(SEQ ID NO: 11)-(SEQ ID NO: 40-(SEQ ID NO: 45)-(SEQ ID NO: 46).

10. The biparatopic antibody of claim 1, further comprising an interleukin-15 (IL-15) domain having at least 85% sequence identity to SEQ ID NO: 47 or having at least 85% sequence identity to SEQ ID NO: 48.

11. The biparatopic antibody of claim 10, wherein the IL-15 domain comprises SEQ ID NO: 47 or SEQ ID NO: 48.

12. The biparatopic antibody of claim 10, wherein the IL-15 domain is conjugated to the C-terminal end of the second arm via a linker.

13. The biparatopic antibody of claim 1, further comprising an IL-15 receptor alpha Sushi (IL15RαSu) domain having at least 85% sequence identity to SEQ ID NO: 49.

14. The biparatopic antibody of claim 13, wherein the IL15RαSu domain comprises SEQ ID NO: 49.

15. The biparatopic antibody of claim 14, wherein the IL15RαSu domain is conjugated to the C-terminal end of the first arm via a linker.

16. The biparatopic antibody of claim 12, wherein:
the first arm has an arrangement of (SEQ ID NO: 37)-(SEQ ID NO: 12)-(SEQ ID NO: 38)-(SEQ ID NO: 5)-(SEQ ID NO: 39)-(SEQ ID NO: 13)-(SEQ ID NO: 40)-linker-(SEQ ID NO: 41)-(SEQ ID NO: 25)-(SEQ ID NO: 42)-(SEQ ID NO: 26)-(SEQ ID NO: 43)-(SEQ ID NO: 31)-(SEQ ID NO: 44)-linker-(SEQ ID NO: 46)-linker-(SEQ ID NO: 49), and
in the second arm, the V_{L} chain has an arrangement of (SEQ ID NO: 41)-(SEQ ID NO: 25-(SEQ ID NO: 42)-(SEQ ID NO: 26)-(SEQ ID NO: 43)-(SEQ ID NO: 30)-(SEQ ID NO: 44) and the V_{H} chain has an arrangement of (SEQ ID NO: 37)-(SEQ ID NO: 4)-(SEQ ID NO: 38)-(SEQ ID NO: 10)-(SEQ ID NO: 39)-(SEQ ID NO: 11)-(SEQ ID NO: 40-(SEQ ID NO: 40)-(SEQ ID NO: 454)-(SEQ ID NO: 46)-linker-(SEQ ID NO: 48).

17. A polynucleotide encoding the biparatopic antibody of claim 1.

18. A pharmaceutical composition comprising the biparatopic antibody of claim 1 and a pharmaceutically acceptable excipient.

19. The pharmaceutical composition of claim 18, wherein the pharmaceutical composition is formulated for intravenous administration.

20. The pharmaceutical composition of claim 18, further comprising a natural killer (NK) cell lysate comprising:
a salt solution;
a cytotoxic protein; and
a cytokine; wherein the cytokine is interleukin-16 (IL-16).

21. The pharmaceutical composition of claim 20, wherein the NK cells are NK-92/aNK cells, haNK cells, primary NK cells, KHYG-1 cells, or a variant thereof.

22. The pharmaceutical composition of claim 18, further comprising one or more additional cytokine selected from the group consisting of IL-1α, IL-1β, IL-2, IL-4, IL-5, IL-6, IL-7, IL-10, IL-12p40, IL-12p70, IL-13, IL-15, IL-17, INF-γ, GM-CSF, TNF-α, TNF-βVEGF, IL-8, Eotaxin-3, MCP-1, MCP-4, MDC, MIP-1α, MIP-1β, and TARC.

23. The pharmaceutical composition of claim 18, further comprising a stabilized IL-15:IL-15Rα.

24. A method of treating cancer in a subject in need thereof, the method comprising administering to the subject the biparatopic antibody according to claim 1 or a pharmaceutical composition comprising the biparatopic antibody.

25. The method of claim 24, wherein the cancer is multiple myeloma.


SEQUENCE LISTING (91 sequences)

SEQ ID NO: 1 - protein, 6 aa, synthetic construct
SSYWMH

SEQ ID NO: 2 - protein, 10 aa, synthetic construct
AIDGNGASTY

SEQ ID NO: 3 - protein, 7 aa, synthetic construct
DLAVFWG

SEQ ID NO: 4 - protein, 6 aa, synthetic construct
SSYAMH

SEQ ID NO: 5 - protein, 10 aa, synthetic construct
AIWGSGGRTY

SEQ ID NO: 6 - protein, 8 aa, synthetic construct
GRSLSRPA

SEQ ID NO: 7 - protein, 6 aa, synthetic construct
SEYAMH

SEQ ID NO: 8 - protein, 10 aa, synthetic construct
AISWSGGYTY

SEQ ID NO: 9 - protein, 7 aa, synthetic construct
DLILFLG

SEQ ID NO: 10 - protein, 10 aa, synthetic construct
AISGSGGSTY

SEQ ID NO: 11 - protein, 12 aa, synthetic construct
DLFPSDMMLFLG

SEQ ID NO: 12 - protein, 6 aa, synthetic construct
SRYAMH

SEQ ID NO: 13 - protein, 9 aa, synthetic construct
LGRTLSRSG

SEQ ID NO: 14 - protein, 10 aa, synthetic construct
AISGGGESTY

SEQ ID NO: 15 - protein, 12 aa, synthetic construct
DLGHHDLWWFSG

SEQ ID NO: 16 - protein, 10 aa, synthetic construct
AIRGSGGSTY

SEQ ID NO: 17 - protein, 12 aa, synthetic construct
DLWILWTVSVSG

SEQ ID NO: 18 - protein, 6 aa, synthetic construct
SGYAMH

SEQ ID NO: 19 - protein, 10 aa, synthetic construct
AISGSGGKTY

SEQ ID NO: 20 - protein, 10 aa, synthetic construct
DLGPWIWGNA

SEQ ID NO: 21 - protein, 10 aa, synthetic construct
AINGNGGSTH

SEQ ID NO: 22 - protein, 11 aa, synthetic construct
DLWEYVLLSFG

SEQ ID NO: 23 - skipped

SEQ ID NO: 24 - protein, 10 aa, synthetic construct
DRVQFSGLGF

SEQ ID NO: 25 - protein, 11 aa, synthetic construct
QASQDISNYLN

SEQ ID NO: 26 - protein, 7 aa, synthetic construct
DASNLET

SEQ ID NO: 27 - protein, 5 aa, synthetic construct
FDSWP

SEQ ID NO: 28 - protein, 6 aa, synthetic construct
LYDLPW

SEQ ID NO: 29 - protein, 6 aa, synthetic construct
DASGPW

SEQ ID NO: 30 - protein, 6 aa, synthetic construct
FYNSPL

SEQ ID NO: 31 - protein, 6 aa, synthetic construct
LYDIPW

SEQ ID NO: 32 - protein, 6 aa, synthetic construct
TASVPL

SEQ ID NO: 33 - protein, 6 aa, synthetic construct
SSASPW

SEQ ID NO: 34 - protein, 6 aa, synthetic construct
DASRPL

SEQ ID NO: 35 - protein, 6 aa, synthetic construct
FYSWPL

SEQ ID NO: 36 - protein, 6 aa, synthetic construct
HYDWPL

SEQ ID NO: 37 - protein, 29 aa, synthetic construct
EVQLVESGGGLVQPGGSLRLSCAASGFTF

SEQ ID NO: 38 - protein, 14 aa, synthetic construct
WVRQAPGKGLEWVS

SEQ ID NO: 39 - protein, 39 aa, synthetic construct
YADSVKGRFTISRDNSKNTLYLQMNSLRAEDTAVYYCAR

SEQ ID NO: 40 - protein, 14 aa, synthetic construct
FDVWGQGTLVTVSS

SEQ ID NO: 41 - protein, 23 aa, synthetic construct
DIQMTQSPSSLSASVGDRVTITC

SEQ ID NO: 42 - protein, 15 aa, synthetic construct
WYQQKPGKAPKLLIY

SEQ ID NO: 43 - protein, 34 aa, synthetic construct
GVPSRFSGSGSGTDFTFTISSLQPEDIATYYCQQ

SEQ ID NO: 44 - protein, 118 aa, synthetic construct
TFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 46 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 48 - protein, 114 aa, synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 49 - protein, 65 aa, synthetic construct
ITCPPPMSVEHADIWVKSYSLYSRERYICNSGFKRKAGTSSLTECVLNKATNVAHWTTPSLKCIR

SEQ ID NO: 50 - protein, 221 aa, synthetic construct
KIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 51 - protein, 19 aa, synthetic construct
GGGGSGGGGSGGGGSGGGG

SEQ ID NO: 52 - protein, 15 aa, synthetic construct
GGGGSKGGGGSKGGG

SEQ ID NO: 53 - protein, 15 aa, synthetic construct
GGGGSGGGGSGGGGS

SEQ ID NO: 54 - protein, 30 aa, synthetic construct
GGGGSGGGGSGGGGSGGGGSGGGGSGGGGS

SEQ ID NO: 55 - protein, 213 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQFDSWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 449 aa, synthetic construct
EVQLVESGGGLVQPGGSLRLSCAASGFTFSSYWMHWVRQAPGKGLEWVSAIDGNGASTYYADSVKGRFTISRDNSKNTLYLQMNSLRAEDTAVYYCARDLAVFWGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQLYDLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 450 aa, synthetic construct
EVQLVESGGGLVQPGGSLRLSCAASGFTFSSYAMHWVRQAPGKGLEWVSAIWGSGGRTYYADSVKGRFTISRDNSKNTLYLQMNSLRAEDTAVYYCARGRSLSRPAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQDASGPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 449 aa, synthetic construct
EVQLVESGGGLVQPGGSLRLSCAASGFTFSEYAMHWVRQAPGKGLEWVSAISWSGGYTYYADSVKGRFTISRDNSKNTLYLQMNSLRAEDTAVYYCARDLILFLGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQFYNSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 454 aa, synthetic construct
EVQLVESGGGLVQPGGSLRLSCAASGFTFSSYAMHWVRQAPGKGLEWVSAISGSGGSTYYADSVKGRFTISRDNSKNTLYLQMNSLRAEDTAVYYCARDLFPSDMMLFLGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQLYDIPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 450 aa, synthetic construct
EVQLVESGGGLVQPGGSLRLSCAASGFTFSRYAMHWVRQAPGKGLEWVSAIWGSGGRTYYADSVKGRFTISRDNSKNTLYLQMNSLRAEDTAVYYCALGRTLSRSGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 214 aa, synthetic construct; identical to SEQ ID NO: 63

SEQ ID NO: 66 - protein, 530 aa, synthetic construct
EVQLVESGGGLVQPGGSLRLSCAASGFTFSRYAMHWVRQAPGKGLEWVSAIWGSGGRTYYADSVKGRFTISRDNSKNTLYLQMNSLRAEDTAVYYCALGRTLSRSGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSITCPPPMSVEHADIWVKSYSLYSRERYICNSGFKRKAGTSSLTECVLNKATNVAHWTTPSLKCIR

SEQ ID NO: 67 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQHYDWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 451 aa, synthetic construct
EVQLVESGGGLVQPGGSLRLGCAASGFTFSSYAMHWVRQAPGKGLEWVSIISGSGGSTYYADSVKGRFTISRDNSKNTLYLQMNSLRAEDTAVYYCARDRVQFSGLGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQTASVPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 454 aa, synthetic construct
EVQLVESGGGLVQPGGSLRLSCAASGFTFSSYAMHWVRQAPGKGLEWVSAISGGGESTYYADSVKGRFTISRDNSKNTLYLQMNSLRAEDTAVYYCARDLGHHDLWWFSGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQSSASPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 454 aa, synthetic construct
EVQLVESGGGLVQPGGSLRLSCAASGFTFSEYAMHWVRQAPGKGLEWVSAIRGSGGSTYYADSVKGRFTISRDNSKNTLYLQMNSLRAEDTAVYYCARDLWILWTVSVSGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 214 aa, synthetic construct
DIQMTQSPSSLSASVGDRVTITCQASQDISNYLNWYQQKPGKAPKLLIYDASNMETGVPSRFSGSGSGTDFTFTISSLQPEDIATYYCQQDASRPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 452 aa, synthetic construct
EVQLVESGGGLVQPGGSLRLSCAASGFTFSGYAMHWVRQAPGKGLEWVSAISGSGGKTYYADSVKGRFTISRDNSKNTLYLQMNSLRAEDTAVYYCARDLGPWIWGNAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQFYSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 453 aa, synthetic construct
EVQLVESGGGLVQPGGSLRLSCAASGFTFSSYAMHWVRQAPGKGLEWVSAINGNGGSTHYADSVKGRFTISRDNSKNTLYLQMNSLRAEDTAVYYCARDLWEYVLLSFGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 488 aa, synthetic construct
EVQLVESGGGLVQPGGSLRLSCAASGFTFSRYAMHWVRQAPGKGLEWVSAIWGSGGRTYYADSVKGRFTISRDNSKNTLYLQMNSLRAEDTAVYYCALGRTLSRSGFDVWGQGTLVTVSSGGGGSGGGGSGGGGSGGGGDIQMTQSPSSLSASVGDRVTITCQASQDISNYLNWYQQKPGKAPKLLIYDASNLETGVPSRFSGSGSGTDFTFTISSLQPEDIATYYCQQLYDIPWTFGQGTKVEIKGGGGSKGGGGSKGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVTLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 214 aa, synthetic construct; identical to SEQ ID NO: 61

SEQ ID NO: 79 - protein, 454 aa, synthetic construct
EVQLVESGGGLVQPGGSLRLSCAASGFTFSSYAMHWVRQAPGKGLEWVSAISGSGGSTYYADSVKGRFTISRDNSKNTLYLQMNSLRAEDTAVYYCARDLFPSDMMLFLGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCVVKGFYPSDIAVEWESNGQPENNYKTTPPVLDSDGSFFLVSKLTVDKSRWQQGNVFSCSVMHEALHNHYTQKSLSLSPGK

SEQ ID NO: 80 - protein, 488 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQLYDIPWTFGQGTKVEIKGGGGSGGGGSGGGGSGGGGEVQLVESGGGLVQPGGSLRLSCAASGFTFSRYAMHWVRQAPGKGLEWVSAIWGSGGRTYYADSVKGRFTISRDNSKNTLYLQMNSLRAEDTAVYYCALGRTLSRSGFDVWGQGTLVTVSSGGGKPGGGKPGGGKPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVTLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 214 aa, synthetic construct; identical to SEQ ID NO: 61

SEQ ID NO: 82 - protein, 454 aa, synthetic construct; identical to SEQ ID NO: 79

SEQ ID NO: 83 - protein, 492 aa, synthetic construct
EVQLVESGGGLVQPGGSLRLSCAASGFTFSSYAMHWVRQAPGKGLEWVSAISGSGGSTYYADSVKGRFTISRDNSKNTLYLQMNSLRAEDTAVYYCARDLFPSDMMLFLGFDVWGQGTLVTVSSGGGGSGGGGSGGGGSGGGGDIQMTQSPSSLSASVGDRVTITCQASQDISNYLNWYQQKPGKAPKLLIYDASNLETGVPSRFSGSGSGTDFTFTISSLQPEDIATYYCQQFYNSPLTFGQGTKVEIKGGGGSKGGGGSKGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVTLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 214 aa, synthetic construct; identical to SEQ ID NO: 63

SEQ ID NO: 85 - protein, 450 aa, synthetic construct
EVQLVESGGGLVQPGGSLRLSCAASGFTFSRYAMHWVRQAPGKGLEWVSAIWGSGGRTYYADSVKGRFTISRDNSKNTLYLQMNSLRAEDTAVYYCALGRTLSRSGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCVVKGFYPSDIAVEWESNGQPENNYKTTPPVLDSDGSFFLVSKLTVDKSRWQQGNVFSCSVMHEALHNHYTQKSLSLSPGK

SEQ ID NO: 86 - protein, 568 aa, synthetic construct
EVQLVESGGGLVQPGGSLRLSCAASGFTFSRYAMHWVRQAPGKGLEWVSAIWGSGGRTYYADSVKGRFTISRDNSKNTLYLQMNSLRAEDTAVYYCALGRTLSRSGFDVWGQGTLVTVSSGGGGSGGGGSGGGGSGGGGDIQMTQSPSSLSASVGDRVTITCQASQDISNYLNWYQQKPGKAPKLLIYDASNLETGVPSRFSGSGSGTDFTFTISSLQPEDIATYYCQQLYDIPWTFGQGTKVEIKGGGGSKGGGGSKGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVTLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSITCPPPMSVEHADIWVKSYSLYSRERYICNSGFKRKAGTSSLTECVLNKATNVAHWTTPSLKCIR

SEQ ID NO: 87 - protein, 214 aa, synthetic construct; identical to SEQ ID NO: 61

SEQ ID NO: 88 - protein, 598 aa, synthetic construct
EVQLVESGGGLVQPGGSLRLSCAASGFTFSSYAMHWVRQAPGKGLEWVSAISGSGGSTYYADSVKGRFTISRDNSKNTLYLQMNSLRAEDTAVYYCARDLFPSDMMLFLGFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCVVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSGGGGSGGGGSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 89 - protein, 470 aa, synthetic construct
EVQLVESGGGLVQPGGSLRLSCAASGFTFSSYAMHWVRQAPGKGLEWVSAISGSGGSTYYADSVKGRFTISRDNSKNTLYLQMNSLRAEDTAVYYCARDLFPSDMMLFLGFDVWGQGTLVTVSSGGGGSGGGGSGGGGSGGGGDIQMTQSPSSLSASVGDRVTITCQASQDISNYLNWYQQKPGKAPKLLIYDASNLETGVPSRFSGSGSGTDFTFTISSLQPEDIATYYCQQFYNSPLTFGQGTKVEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 90 - protein, 466 aa, synthetic construct
EVQLVESGGGLVQPGGSLRLSCAASGFTFSRYAMHWVRQAPGKGLEWVSAIWGSGGRTYYADSVKGRFTISRDNSKNTLYLQMNSLRAEDTAVYYCALGRTLSRSGFDVWGQGTLVTVSSGGGGSGGGGSGGGGSGGGGDIQMTQSPSSLSASVGDRVTITCQASQDISNYLNWYQQKPGKAPKLLIYDASNLETGVPSRFSGSGSGTDFTFTISSLQPEDIATYYCQQLYDIPWTFGQGTKVEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 91 - protein, 7 aa, synthetic construct
DASNMET